(19) United States
(12) Patent Application Publication
(10) Pub. No.: US 2012/0184586 A1
(43) Pub. Date: Jul. 19, 2012

(54) DESFERRITHIOCIN POLYETHER ANALOGUES AND USES THEREOF

(75) Inventor: Raymond J. Bergeron, JR., Gainesville, FL (US)

(73) Assignee: University of Florida Research Foundation, Inc., Gainesville, FL (US)

(21) Appl. No.: 13/390,951

(22) PCT Filed: Aug. 25, 2010
(86) PCT No.: PCT/US10/02336

§ 371 Date: Mar. 30, 2012

Related U.S. Application Data
(60) Provisional application No. 61/275,096, filed on Aug. 25, 2009.

(51) Int. Cl. A61K 31/426; A61P 39/06; A61P 39/04; C07D 277/12

(52) U.S. Cl. 514/365; 548/201

(57) ABSTRACT

Desferrithiocin analogues represents by the structural formulae described here, such as formula (I), are useful in treating conditions such as metal overload (e.g., iron overload from transfusion therapy), oxidative stress, and neoplastic and preneoplastic conditions.

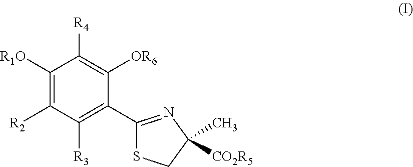

### RELATED APPLICATIONS

The present application claims priority under 35 U.S.C. §119(e) to U.S. provisional patent application, U.S. Ser. No. 61/275,096, filed Aug. 25, 2009, which is incorporated herein by reference.

### GOVERNMENT SUPPORT

The invention was made with U.S. Government support under grant no. 5 R37 DK049108 from the National Diabetes and Digestive and Kidney Diseases Advisory Council (NIDDK) of the National Institutes of Health (NIH). The U.S. Government has certain rights in the invention.

### BACKGROUND OF THE INVENTION

Iron metabolism in primates is characterized by a highly efficient recycling process. There is no specific mechanism for eliminating this transition metal. Because of the lack of an iron clearance mechanism, the introduction of “excess iron” into primates often leads to chronic overload and can ultimately lead to biological damage (e.g., peroxidative tissue damage). There are a number of ways in which excess iron is introduced, including a high-iron diet, acute iron ingestion, or malabsorption of the metal. In each of these situations, a subject can typically be treated by phlebotomy to reduce iron levels. However, for iron overload syndromes resulting from chronic transfusion therapy, e.g., aplastic anemia and thalassemia, phlebotomy is not an option. In these secondary iron overload syndromes, the origin of the excess iron is the transfused red blood cells. Since removing the red blood cells to remedy the iron overload would be counterproductive, an alternative approach to removing iron is chelation therapy. Although considerable effort has been invested in the development of new therapeutics for managing iron overload in patients with thalassemia, particularly therapeutics that can be administered orally, desferrioxamine B, a hexacoordinate hydroxamate iron chelator produced by Streptomyces pilosus, is still the agent of choice. However, desferrioxamine B is not ideal for chelation therapy because iron is removed with a low efficiency. In addition, the oral activity of desferrioxamine B is marginal, thereby requiring parenteral administration, which can result in poor patient compliance, particularly for patients in need of long-term chelation therapy. A substantial number of synthetic iron chelators have been studied in recent years as potential orally active therapeutics, e.g., pyridoxal isonicotinoyl hydrazone (PIH), hydroxypyridones, and N,N′-bis-(2-hydroxybenzylethylenediamine)-N,N′-diacetic acid (HBED); however, these synthetic chelators have not yet demonstrated the desired properties (e.g., effective chelation, suitable oral activity, and acceptable toxicity). Siderophores including enterobactin and rhodotorulic acid have also been studied for chelation therapy. However, both enterobactin and rhodotorulic acid have exhibited unacceptable toxicity, and neither demonstrates measurable oral activity. In general, although a large number of siderophores and synthetic iron chelators have been developed, most have been abandoned because their properties are not suitable for use in treating chronic iron overload.

Therefore, a need still exists for novel iron chelators that can be used in chelation therapy, especially chronic chelation therapy. Preferably chelators for use in treating iron overload in a subject need to be efficient in chelating and removing iron from an organism, possess suitable oral bioavailability, and/or pose minimal toxicity to a subject.

### SUMMARY OF THE INVENTION

Compounds are provided which are useful as metal chelators. These compounds may be useful in treating a disease associated with the accumulation of metals in a subject (e.g., chronic transfusion therapy associated with the treatment of thalassemia or other transfusion-dependent anemias, acute iron ingestion, etc.). Previously, certain desferrithiocin polyether analogues were described in published international PCT application, WO 2006/107626, published Oct. 12, 2006; which is incorporated herein by reference. It has been discovered by the inventors that the shorter polyether chain of the compounds of the present invention lead to solid forms, rather than oils. In certain embodiments, the purified inventive compound is a solid, including a crystalline solid.

In certain embodiments, the compound is of the formula (I):

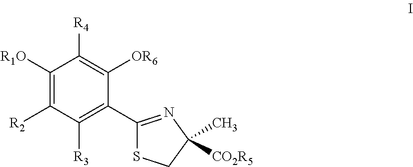

wherein

R₁ is —[(CH₂)ₙ—O]ₓ—R′;

R₂, R₃, and R₄ are each independently —H, an alkyl group, or —OR₇;

R₅ is —H or an alkyl group;

R₆ is —H, an alkyl group, an O-protecting group, or an acyl group;

each R₇ is independently —H, an alkyl group, an O-protecting group, or an acyl group;

R′ is —H, an alkyl group, an O-protecting group, or an acyl group;

each n is 2;

x is 1 or 2; or a salt, solvate or hydrate thereof;

with the proviso that the compound of formula (I) is not of formula (II):

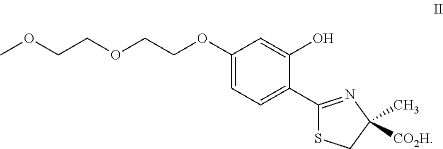

In any of the embodiments described herein, the compound can be a solid, including a crystalline solid.

In certain embodiments, the length of the polyethylene glycol chain is 8 carbon and oxygen atoms long. In other embodiments, the length of said chain is of 5 carbon and oxygen atoms long. In certain embodiments, the compound is a carboxylic acid, methyl ester, ethyl ester, propyl ester, or iso-propyl ester. In certain embodiments, the compound is a carboxylic acid. In certain embodiments, the compound is a methyl ester. In certain embodiments, the compound is an ethyl ester.

In certain embodiments, R₆ is hydrogen. In certain embodiments, all of R₂, R₃, and R₄ are hydrogen. In certain embodiments, R₅ is hydrogen. In certain embodiments, R₅ is methyl. In certain embodiments, R₅ is ethyl. In certain embodiments, R₅ is propyl. In certain embodiments, R₅ is iso-propyl.

In certain embodiments, the compound is:

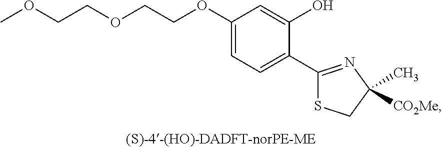

or a salt, solvate, or hydrate thereof.

In certain embodiments, the compound is:

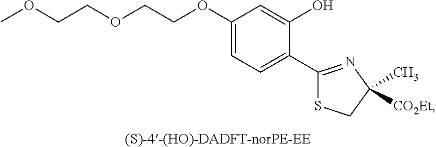

or a salt, solvate, or hydrate thereof.

In certain embodiments, the compound is:

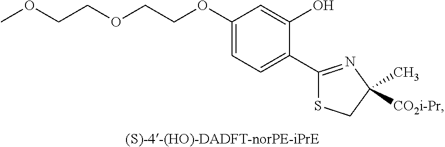

or a salt, solvate, or hydrate thereof.

In other embodiments, the compound is a solid form of:

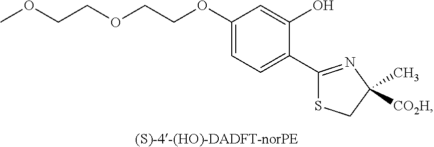

or a salt, solvate, or hydrate thereof.

In certain embodiments, the compound is a crystalline form of:

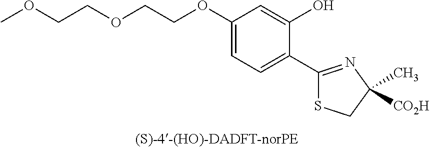

or a salt, solvate, or hydrate thereof.

The metal chelators of the invention have the advantage of having a desirable iron clearing efficiency. The metal chelators of the invention can possess a different volume of distribution from known chelators, resulting in a different distribution among organs. This different distribution can permit penetration into organs such as the heart, brain, and pancreas, as well as result in the majority of clearance of the chelator by the liver, thereby decreasing the risk of renal toxicity.

The invention also provides pharmaceutical compositions comprising a therapeutically effective amount of a compound of the invention and a pharmaceutically acceptable excipient. The pharmaceutical compositions are useful in treating iron overload.

In another embodiment, the present invention is a method of treating a pathological condition responsive to chelation of a trivalent metal (e.g. Fe³⁺) in a subject, comprising administering to the subject a therapeutically or prophylactically effective amount of a compound, or a pharmaceutical composition thereof. In certain embodiments, the compound or pharmaceutical composition is administered orally. In other embodiments, the compound or pharmaceutical composition is administered parenterally (e.g., intravenously).

The compounds of the invention can also be used in a method of reducing oxidative stress in a subject and a method of treating a subject who is suffering from neoplastic disease or a preneoplastic condition, in which a therapeutically effective amount of an inventive compound, or a pharmaceutical composition thereof, is administered to the subject.

The invention also relates to the use of compounds disclosed herein in the treatment of diseases or disorders associated with metal overload, oxidative stress, and neoplastic and preneoplastic conditions. In certain embodiments, the disease or disorder is associated with iron overload.

The invention further relates to the use of the compounds of the invention for the manufacture of a medicament for treating pathological conditions responsive to chelation or sequestration of metals, for reducing oxidative stress, or for treating neoplastic disease or a pre-neoplastic condition.

### DEFINITIONS

Before further description of the present invention, and in order that the invention may be more readily understood, certain terms are first defined and collected here for convenience.

Certain compounds of the present invention, and definitions of specific functional groups are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

One of ordinary skill in the art will appreciate that the compounds and synthetic methods, as described herein, utilize a variety of protecting groups. By the term “protecting group”, has used herein, it is meant that a particular functional moiety, e.g., C, O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative disorders, including, but not limited to cancer. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

An alkyl group is a saturated hydrocarbon in a molecule that is bonded to one other group in the molecule through a single covalent bond from one of its carbon atoms. Alkyl groups can be cyclic or acyclic, branched or unbranched (straight chained) and substituted or unsubstituted when straight chained or branched. An alkyl group typically has from 1 to about 12 carbon atoms, for example, one to about six carbon atoms or one to about four carbon atoms. Lower alkyl groups have one to four carbon atoms and include methyl, ethyl, n-propyl, iso-propyl, n-butyl, sec-butyl and tert-butyl. When cyclic, an alkyl group typically contains from about 3 to about 10 carbons, for example, from about 3 to about 8 carbon atoms, e.g., a cyclopropyl group, a cyclobutyl group, a cyclopentyl group, a cyclohexyl group, a cycloheptyl group or a cyclooctyl group.

An alkoxy group is an alkyl group, as previously defined, attached to the parent molecular moiety through an oxygen atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

Acyl groups are represented by the formula —C(O)R, where R is an alkyl group. Acyl groups can be hydrolyzed or cleaved from a compound by enzymes, acids, or bases. One or more of the hydrogen atoms of an acyl group can be substituted, as described below. Typically, an acyl group is removed before a compound of the present invention binds to a metal ion such as iron(III).

Suitable substituents for alkyl and acyl groups include —OH, —O(R″), —COOH, ═O, —NH₂, —NH(R″), —NO₂, —COO(R″), —CONH₂, —CONH(R″), —CON(R″)₂, and guanidine. Each R″ is independently an alkyl group or an aryl group. These groups can additionally be substituted by an aryl group (e.g., an alkyl group can be substituted with an aromatic group to form an arylalkyl group). A substituted alkyl or acyl group can have more than one substituent.

Aryl groups include carbocyclic aromatic groups such as phenyl, p-tolyl, 1-naphthyl, 2-naphthyl, 1-anthracyl and 2-anthracyl. Aryl groups also include heteroaromatic groups such as N-imidazolyl, 2-imidazolyl, 2-thienyl, 3-thienyl, 2-furanyl, 3-furanyl, 2-pyridyl, 3-pyridyl, 4-pyridyl, 2-pyrimidyl, 4-pyrimidyl, 2-pyranyl, 3-pyranyl, 3-pyrazolyl, 4-pyrazolyl, 5-pyrazolyl, 2-pyrazinyl, 2-thiazolyl, 4-thiazolyl, 5-thiazolyl, 2-oxazolyl, 4-oxazolyl and 5-oxazolyl.

Aryl groups also include fused polycyclic aromatic ring systems in which a carbocyclic, alicyclic, or aromatic ring or heteroaryl ring is fused to one or more other heteroaryl or aryl rings. Examples include 2-benzothienyl, 3-benzothienyl, 2-benzofuranyl, 3-benzofuranyl, 2-indolyl, 3-indolyl, 2-quinolinyl, 3-quinolinyl, 2-benzothiazolyl, 2-benzoxazolyl, 2-benzimidazolyl, 1-isoquinolinyl, 3-isoquinolinyl, 1-isoindolyl, and 3-isoindolyl.

The term “O-protecting group” means a substituent which protects hydroxyl groups against undesirable reactions during synthetic procedures. Examples of O-protecting groups include, but are not limited to, methoxymethyl, benzyloxymethyl, 2-methoxyethoxymethyl, 2-(trimethylsilyl)ethoxymethyl, benzyl, triphenylmethyl, 2,2,2-trichloroethyl, t-butyl, trimethylsilyl, t-butyldimethylsilyl, t-butyldiphenylsilyl, methylene acetal, acetonide benzylidene acetal, cyclic ortho esters, methoxymethylene, cyclic carbonates, and cyclic boronates.

The term “leaving group” refers to a molecular fragment that can departs with a pair of electrons in heterolytic bond cleavage. Examples of leaving groups include, but are not limited to, halides, such as Br, Cl, I; sulfonates, such as tosylates, nosylates, myselates; nonaflates; triflates; fluorosulfonates; nitrates; and phosphates.

Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

### BRIEF DESCRIPTION OF THE DRAWINGS

FIG. 1 illustrates the iron-clearing efficiency of desferrithiocin analogues administered orally to rodents and primates with the respective Log Pₐₚₚ values and physiochemical properties. ^{a}In the rodents [n=3 (6), 4 (3-5, 7), 5 (1), 8 (2)], the drugs were given po at a dose of 150 μmol/kg (1) or 300 μmol/kg (2-7). The drugs were administered in capsules (6, 7), solubilized in either 40% Cremophor RH-40/water (1), distilled water (4), or were given as their monosodium salts, prepared by the addition of 1 equiv of NaOH to a suspension of the free acid in distilled water (2, 3, 5). The efficiency of each compound was calculated by subtracting the 24-h iron excretion of control animals from the iron excretion of the treated animals. The number was then divided by the theoretical output; the result is expressed as a percent. The ICE data for ligand 1 is from ref 39. The ICE data for 2-4 are from ref 34. ^{b}ICE is based on a 48 h sample collection period. The relative percentages of the iron excreted in the bile and urine are in brackets. ^{c}In the primates [n=4 (1, 3, 4, 5, 6 in capsules, 7) or 7 (2, 6 as the monosodium salt)], the chelators were given po at a dose of 75 μmol/kg (5-7) or 150 μmol/kg (1-4). The drugs were administered in capsules (6^{d}, 7), solubilized in either 40% Cremophor RH-40/water (1, 3), distilled water (4), or were given as their monosodium salts, prepared by the addition of 1 equiv of NaOH to a suspension of the free acid in distilled water (2, 5, 6^{e}). The efficiency was calculated by averaging the iron output for 4 days before the drug, subtracting these numbers from the 2-day iron clearance after the administration of the drug, and then dividing by the theoretical output; the result is expressed as a percent. The ICE data for ligand 1 is from ref. 40, 41. The ICE data for 2-4 are from ref 42, 43 and 34, respectively. The relative percentages of the iron excreted in the feces and urine are in brackets. ^{f}Performance ratio is defined as the mean ICEₚᵣᵢₘₐₜₑₛ/ICE_{rodents}. ^{g}Data are expressed as the log of the fraction in the octanol layer (log Pₐₚₚ); measurements were done in TRIS buffer, pH 7.4, using a “shake flask” direct method.⁵² The values for 2 and 3 are from ref. 43; the value for 4 is from ref. 34. ^{h}The mp data for 1-3 are from ref. 39, 42, and 43, respectively.

FIG. 2 illustrates the iron clearance induced by Desferrithiocin-related chelators in non-iron-loaded, bile duct-cannulated rats (300 μmol/kg PO).

FIG. 3 illustrates the iron tissue concentrations in the organs of rats. FIG. 3a illustrates the iron tissue concentrations in rats treated with (S)-4′-(HO)-DADFT-norPE-EE, while FIG. 3b illustrates the iron tissue concentrations in the corresponding age-matched controls.

FIG. 4 represents the iron tissue concentrations in the organs of rats treated with (S)-4′-(HO)-DADFT-norPE-acid or ethyl ester and control rats over 10 days (384 μmol/kg/d).

FIG. 5 represents the iron tissue concentrations in the organs of rats treated with (S)-4′-(HO)-DADFT-norPE-ethyl ester and control rats over 10 days (192 or 384 μmol/kg/d PO).

FIG. 6 illustrates iron excretion in rat (single dose value) using (S)-4′-(HO)-DADFT-PE (dose: 119.85 mg/kg; application: PO; vehicle: dH₂O). FIG. 6a illustrates the clearance iron excretion by bile; FIG. 6b illustrates the cumulative iron excretion by bile; FIG. 6c represents the iron excretion after 48 hours in the urine and in the bile.

FIG. 7 illustrates iron excretion in rat (single dose value) using (S)-4′-(HO)-DADFT-norPE Acid (dose: 106.5 mg/kg; application: PO; vehicle: capsule). FIG. 7a illustrates the clearance iron excretion by bile; FIG. 7b illustrates the cumulative iron excretion by bile; FIG. 7c represents the iron excretion after 48 hours in the urine and in the bile.

FIG. 8 illustrates iron excretion in rat (single dose values) using (S)-4′-(HO)-DADFT-norPE-EE (dose: 115.04 mg/kg; application: PD; vehicle: capsule). FIG. 8a illustrates the clearance iron excretion by bile; FIG. 8b illustrates the cumulative iron excretion by bile; FIG. 8c represents the iron excretion after 48 hours in the urine and in the bile.

FIG. 9 illustrates iron excretion in rat (single dose values) using (S)-4′-(HO)-DADFT-homoPE (dose: 133 mg/kg; vehicle: dH₂O). FIG. 9a illustrates the clearance iron excretion by bile; FIG. 9b illustrates the cumulative iron excretion by bile; FIG. 9c represents the iron excretion after 48 hours in the urine and in the bile.

FIG. 10 illustrates iron excretion in iron-loaded Cebus monkey model (single dose values) using (S)-4′-(HO)-DADFT-PE (drug/Fe: 2; dose: 59.9 mg/kg; vehicle: dH₂O; route: PO). FIG. 10a illustrates the clearance iron excretion by bile; FIG. 10b illustrates the cumulative iron excretion by bile; FIG. 10c represents the induced iron excretion during the first 48 hours post drug in the urine and feces.

FIG. 11 illustrates iron excretion in Fe loaded Cebus monkey model (single dose values) using 4′-norPE acid (drug/Fe: 2; dose: 26.6 mg/kg; vehicle: capsule; route: PO). FIG. 11a illustrates the clearance iron excretion by bile; FIG. 11b illustrates the cumulative iron excretion by bile; FIG. 11c represents the induced iron excretion during the first 48 hours post drug in the urine and feces.

FIG. 12 illustrates iron excretion in Fe loaded Cebus monkey model (single dose values) using 4-norPE acid (drug/Fe: 2; dose: 26.6 mg/kg; vehicle: dH₂O/NaOH; route: PO). FIG. 12a illustrates the clearance iron excretion by bile; FIG. 12b illustrates the cumulative iron excretion by bile; FIG. 12c represents the induced iron excretion during the first 48 hours post drug in the urine and feces.

FIG. 13 illustrates iron excretion in Fe loaded Cebus monkey model (single dose values) using 4′-norPE acid (drug/Fe: 2; dose: 26.6 mg/kg; vehicle: dH₂O/NaOH; route: PO). FIG. 13a illustrates the clearance iron excretion by bile; FIG. 13b illustrates the cumulative iron excretion by bile; FIG. 13c represents the induced iron excretion during the first 48 hours post drug in the urine and feces.

FIG. 14 illustrates iron excretion in Fe loaded Cebus monkey model (single dose values) using 4′-norPE-EE (drug/Fe: 2; dose: 28.8 mg/kg; vehicle: capsule; route: PO). FIG. 14a illustrates the clearance iron excretion by bile; FIG. 14b illustrates the cumulative iron excretion by bile; FIG. 14c represents the induced iron excretion during the first 48 hours post drug in the urine and feces.

FIG. 15 illustrates the X-ray data of (S)-4,5-dihydro-2-[2-hydroxy-4-(3,6-dioxaheptyloxy)phenyl]-4-methyl-4-thiazolecarboxylic acid (6). Structure is drawn at 50% probability ellipsoids.

FIG. 16 illustrates the X-ray data of ethyl (S)-4,5-dihydro-2-[2-hydroxy-4-(3,6-dioxaheptyloxy)phenyl]-4-methyl-4-thiazolecarboxylate (7). Structure is drawn at 50% probability ellipsoids.

### DETAILED DESCRIPTION OF THE INVENTION

This application relates to compounds characterized by the structural formula (I):

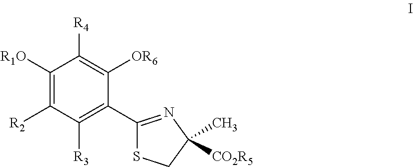

wherein

R₁ is —[(CH₂)ₙ—O]ₓ—R′;

R₂, R₃, and R₄ are each independently —H, an alkyl group, or —OR₇;

R₅ is —H or an alkyl group;

R₆ is —H, an alkyl group, an O-protecting group, or an acyl group;

each R₇ is independently —H, an alkyl group, an O-protecting group, or an acyl group;

R′ is —H, an alkyl group, an O-protecting group, or an acyl group;

each n is 2;

x is 1 or 2;

or a salt, solvate or hydrate thereof.

In certain embodiments, the compound is not of formula (II):

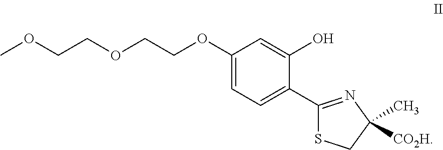

In certain embodiments, the compound is a solid. In other embodiments, the compound is a crystalline solid. In certain embodiments, the compound is an amorphous solid.

In certain embodiments, the compounds of the invention have an enantiomeric excess greater than 80%. In other embodiments, the enantiomeric excess is greater than 90%. In further embodiments, the enantiomeric excess is greater than 95%. In still further embodiments, the enantiomeric excess is greater than 98%. In certain embodiments, the enantiomeric excess is greater than 99%. In specific embodiments, the enantiomeric excess is greater than 99.5%.

As discussed herein and as would be appreciated by one of skill in the art, stereoisomers and mixtures of stereoisomers of the compounds disclosed herein are considered to be within the scope of the invention.

Typically, compounds of the invention are represented by formula (I), where the variables are as disclosed in the genera, classes, subclasses, and species described herein.

In certain embodiments, R₂, R₃, and R₄ are each independently hydrogen, a C₁₋₆ alkyl group, an O-protecting group, or —OR₇; wherein R₇ is hydrogen, a C₁₋₆ alkyl group, an O-protecting group, or an acyl group. In other embodiments, R₂, R₃, and R₄ are each independently hydrogen, a C₁₋₄ alkyl group, or —OR₇; wherein 7₆ is hydrogen, a C₁₋₄ alkyl group, or an acyl group. In other embodiments, R₂, R₃, and R₄ are each independently hydrogen or a C₁₋₄ alkyl group.

In certain embodiments, R₂, R₃, and R₄ are each —H. In other embodiments, R₂, R₃, and R₄ are each independently —H, or a C₁₋₆ alkyl group. In yet other embodiments, R₂, R₃, and R₄ are each independently a methyl, ethyl, propyl, or butyl group. In specific embodiments, R₂, R₃, and R₄ are the same C₁₋₆ alkyl group. In other embodiments, at least on R₂, R₃, or R₄ is methyl. In still other embodiments, at least one R₂, R₃, or R₄ is ethyl. In further embodiments, at least one R₂, R₃, and R₄ is propyl. In specific embodiments, at least one R₂, R₃, and R₄ is butyl. In specific embodiments, R₂, R₃, and R₄ are each hydrogen.

In certain embodiments, at least one R₂, R₃, or R₄ is —OR₇; each R₇ is —H, a C₁₋₄ alkyl group, or an acyl group. In further embodiments, R₇ is —H. In other embodiments, R₇ is a C₁₋₆ alkyl group. In further embodiments, R₇ is an O-protecting group. In still further embodiments, R₇ is an acyl group. In specific embodiments, R₇ is an acetyl group. In other embodiments, R₂, R₃, and R₄ are the same —OR₇.

In certain embodiments, R₆ is —H, an O-protecting group, or an acyl group. In other embodiments, R₆ is —H. In certain embodiments, R₆ is an alkyl group. In certain embodiments, R₆ is a C₁₋₆ alkyl group. In certain embodiments, R₆ is a C₁₋₄ alkyl group. In certain embodiments, R₆ is methyl. In certain embodiments, R₆ is ethyl. In certain embodiments, R₆ is propyl. In certain embodiments, R₆ is buytl. In further embodiments, R₆ is an O-protecting group. In still further embodiments, R₆ is an acyl group. In other embodiments, R₆ is an acetyl group.

In certain embodiments, R₂, R₃, R₄ and R₆ are the same. In other embodiments, R₂, R₃, R₄ and R₆ are each —H. In further embodiments, R₂, R₃, R₄ and R₆ are different. In still further embodiments, R₂ and R₆ are the same. In certain embodiments, R₃ and R₆ are the same. In other embodiments, R₄ and R₆ are the same.

In certain embodiments, x is 1 or 2. In other embodiments, x is 1. In further embodiments, x is 2.

In certain embodiments, R′ is hydrogen. In certain embodiments, R′ is an alkyl group. In other embodiments, R′ is a C₁₋₆ alkyl group. In further embodiments, R′ is a C₁₋₄ alkyl group. In sill further embodiments, R′ is methyl. In other embodiments, R′ is ethyl. In certain embodiments, R′ is propyl. In further embodiments, R′ is butyl.

In certain embodiments, the compounds of the invention are of the formula:

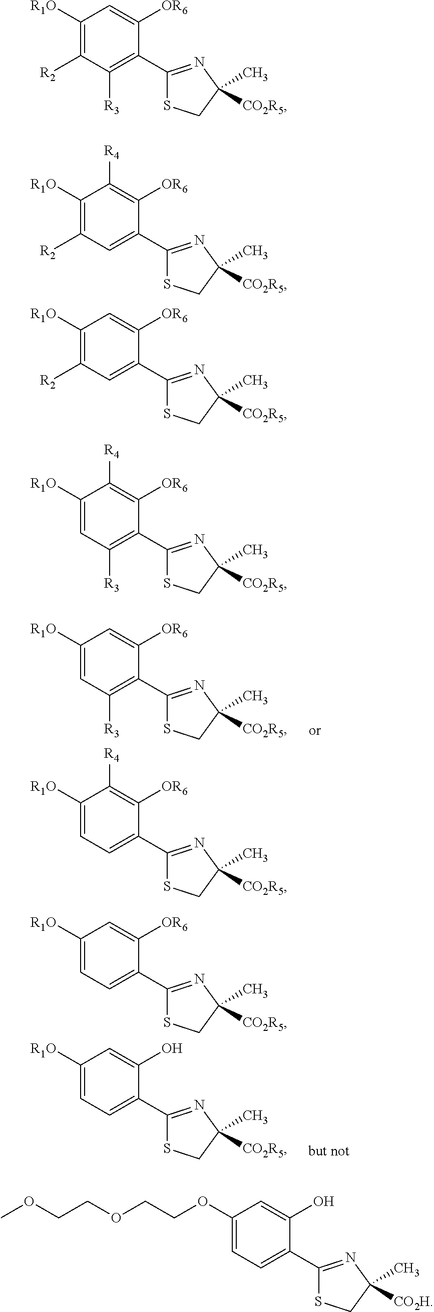

In other embodiments, the compounds of the invention are of the formula:

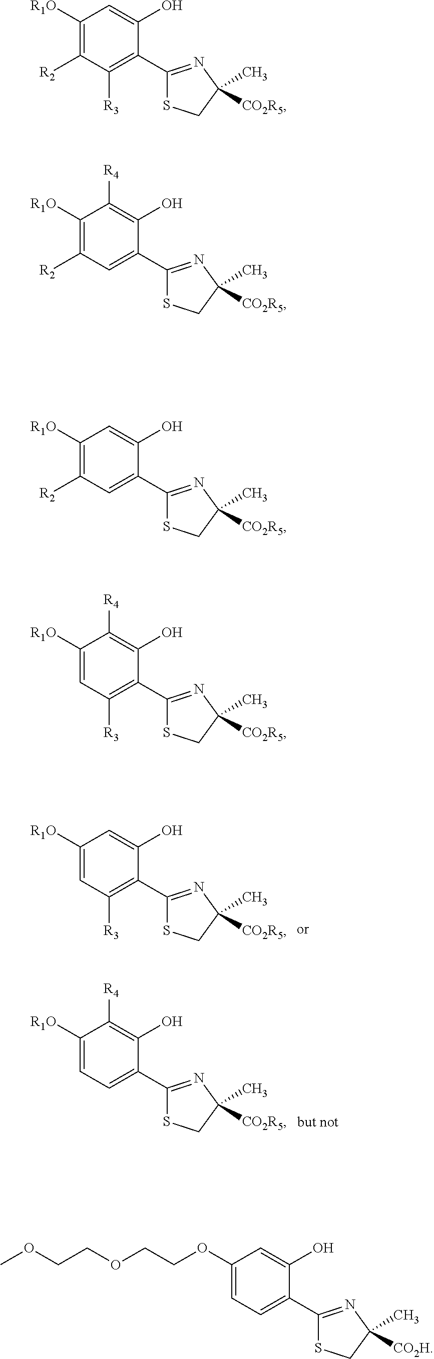

In certain embodiments, the compounds of the invention are of the formula:

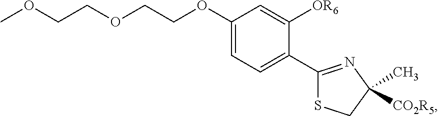

but not

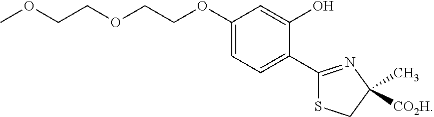

In specific embodiments, the compounds of the invention are of the formula:

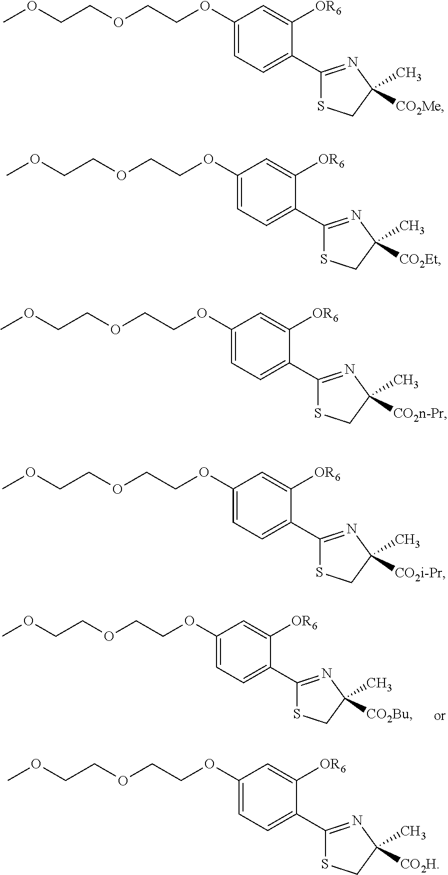

In other specific embodiments, the compound of the invention is:

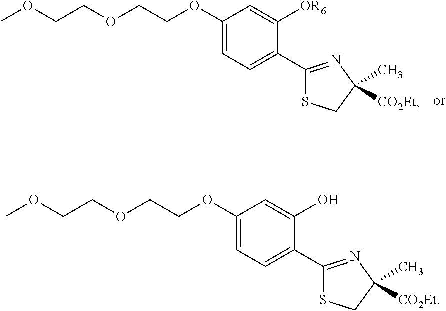

In other embodiments, the inventive compounds have the formula:

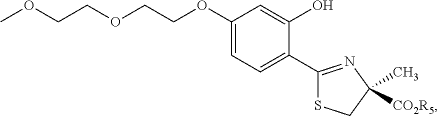

but not

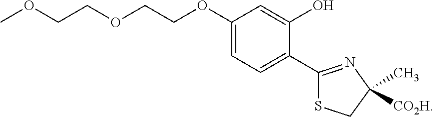

In further embodiments, the inventive compounds have the formula:

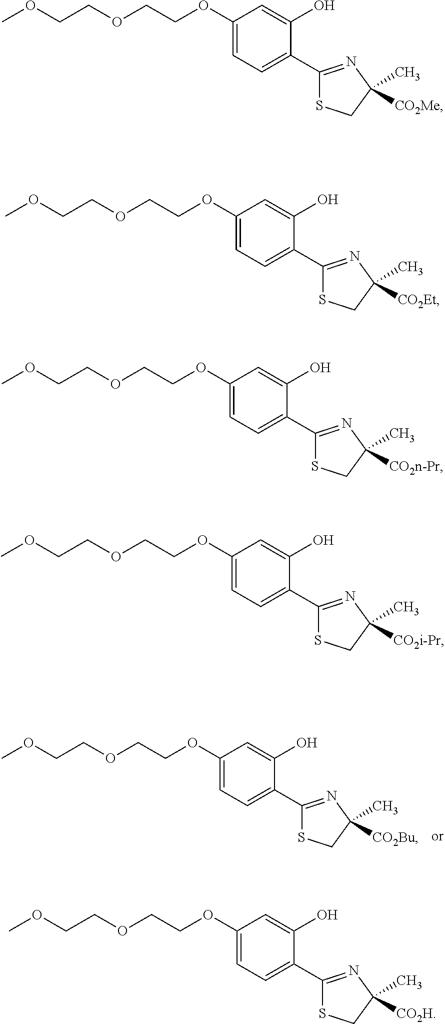

In certain embodiments, the compounds of the invention have the formula:

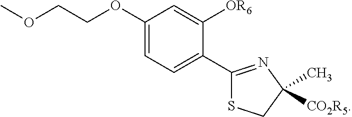

In specific embodiments, the compounds of the invention have the formula:

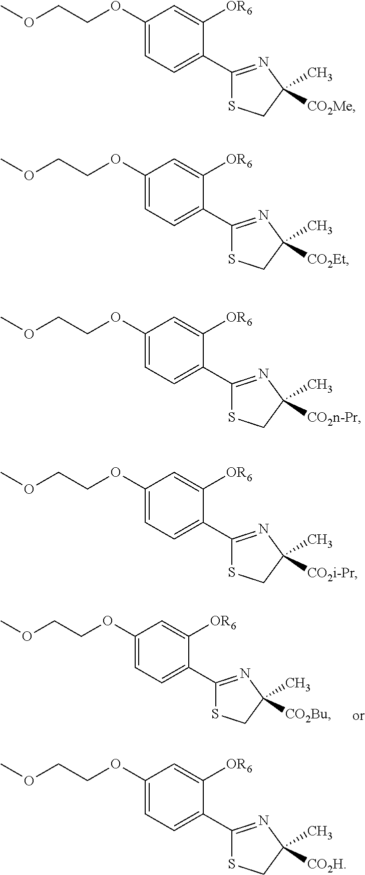

In other embodiments, the inventive compounds have the formula:

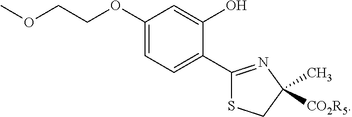

In further embodiments, the inventive compounds have the formula:

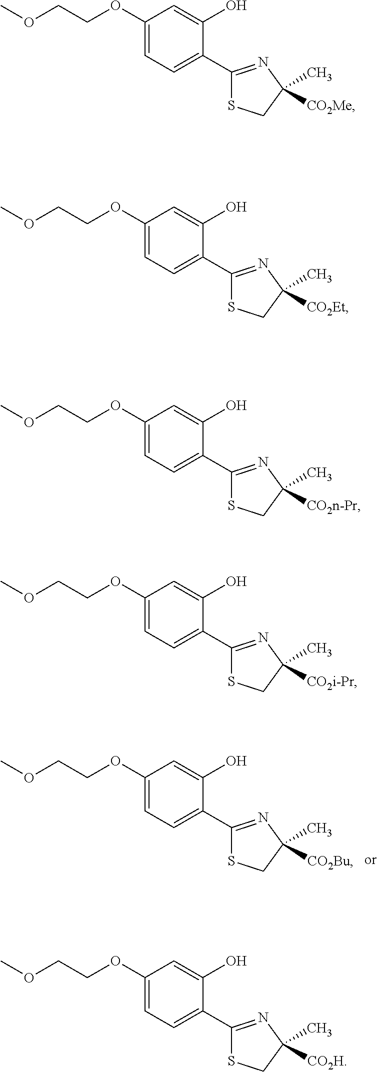

In certain embodiments, the invention provides a solid form of the compound of formula:

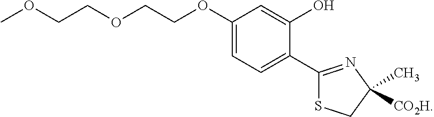

In other embodiments, the inventive compound is a crystalline form of:

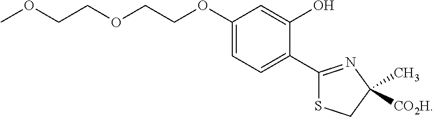

In certain embodiments, the compounds are in salt form. In other embodiments, the salt is a sodium salt. In other embodiments, the salt is a potassium salt. In certain embodiments, the salt is an aluminum salt. In certain embodiments, the salt is a calcium salt.

In certain embodiments, the salt is a lithium salt. In certain embodiments, the salt is a magnesium salt. In certain embodiments, the salt is a barium salt. In other embodiments, the salt is a zinc salt.

In other embodiments, the inventive compound is a salt form of the compound of

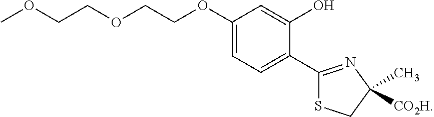

In specific embodiments, the invention provides a composition comprising a compound of formula:

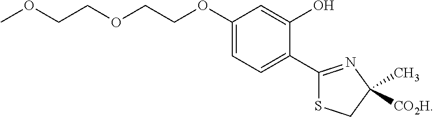

The invention also includes enantiomers and mixtures of enantiomers (e.g., racemic mixtures) of the compounds of the invention, along with their salts (e.g., pharmaceutically acceptable salts), co-crystals, solvates, hydrates, and pro-drugs.

In addition compounds of the invention can exist in optically active forms that have the ability to rotate the plane-polarized light. In describing an optically active compound, the prefixes D and L, or R and S are used to denote the absolute configuration of the substituents about the chiral center. The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that one or more chiral carbons are non-superimposable mirror images of one another. A specific stereoisomer, which is an exact mirror image of another stereoisomer, can also be referred to as an enantiomer, and a mixture of such isomers is often called an enantiomeric mixture. A 50:50 mixture of enantiomers is referred to as a racemic mixture.

As is used in the art, when it is desired to specify the absolute configuration about a chiral carbon, a bond to the chiral carbon can be depicted as a wedge (bonds to atoms above the plane of the paper) and another can be depicted as a series or wedge of short parallel lines (bonds to atoms below the plane of the paper). The Cahn-Ingold-Prelog system can be used to assign the (R) or (S) configuration to a chiral carbon. The chiral carbon at the 4-position of a thiazoline or thiazolidine ring preferably has an (S) configuration.

When compounds of the present invention contain one chiral center, compounds not prepared by an asymmetric synthesis exist in two enantiomeric forms and the present invention includes either or both enantiomers and mixtures of enantiomers, such as the specific 50:50 mixture referred to as a racemic mixture. The enantiomers can be resolved by methods known to those skilled in the art, for example, by formation of diastereoisomeric salts that may be separated, for example, by crystallization (see CRC Handbook of Optical Resolutions via Diastereomeric Salt Formation by David Kozma (CRC Press, 2001)); formation of diastereoisomeric derivatives or complexes that may be separated, for example, by crystallization, gas-liquid or liquid chromatography; selective reaction of one enantiomer with an enantiomer-specific reagent, for example, enzymatic esterification; or gas-liquid or liquid chromatography in a chiral environment, for example, on a chiral support (e.g., silica with a bound chiral ligand) or in the presence of a chiral solvent. It will be appreciated that where the desired enantiomer is converted into another chemical entity by one of the separation procedures described above, a further step is required to liberate the desired enantiomeric form.

Alternatively, specific enantiomers may be synthesized by asymmetric synthesis using optically active reagents, substrates, catalysts, or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or, in other words, is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50% in an enantiomeric mixture. For example, when a mixture contains 80% of a first enantiomer and 20% of a second enantiomer, the enantiomeric excess of the first enantiomer is 60%. In the present invention, the enantiomeric excess can be about 20% or more, particularly about 40% or more, more particularly about 60% or more, such as about 70% or more, for example about 80% or more, such as about 90% or more. In a particular embodiment, the enantiomeric excess of depicted compounds is at least about 90%. In a more particular embodiment, the enantiomeric excess of the compounds is at least about 95%, such as at least about 96%, 97%, 97.5%, 98%, for example, at least about 99% enantiomeric excess.

Also included in the present invention are salts and pharmaceutically acceptable salts of the compounds described herein. Compounds disclosed herein that possess a sufficiently acidic functional group (e.g., a carboxylic acid group), a sufficiently basic functional group, or both, can react with a number of organic or inorganic bases, and inorganic and organic acids, to form salts.

Acidic groups can form salts with one or more of the metals listed above, along with alkali and alkaline earth metals (e.g., sodium, potassium, magnesium, calcium). In addition, acidic groups can form salts with amines. Compounds of the invention can be supplied as a transition, lanthanide, actinide or main group metal salt. As a transition, lanthanide, actinide, or main group metal salt, compounds of the invention tend to form a complex with the metal. For example, if a compound of the invention is tridentate and the metal it forms a salt with has six coordinate sites, then a 2 to 1 compound to metal complex is formed. The ratio of compound to metal will vary according to the density of the metal and the number of coordination sites on the metal (preferably each coordination site is filled by a compound of the invention, although a coordination site can be filled with other anions such as hydroxide, halide, or a carboxylate).

Alternatively, the compound can be a substantially metal-free (e.g. iron-free) salt. Metal-free salts are not typically intended to encompass alkali and alkali earth metal salts.

Metal-free salts are advantageously administered to a subject suffering from, for example, a metal overload condition or to an individual suffering from toxic metal exposure or from focal concentrations of metals causing untoward effects

The inventive compounds and the salts forms thereof can be prepared in the form of their hydrates, such as hemihydrate, monohydrate, dihydrate, trihydrate, tetrahydrate and the like. Solvates such as alcoholates may also be prepared of the inventive compounds.

### Pharmaceutical Compositions

In another aspect of the present invention, pharmaceutical compositions are provided, which comprise any one of the compounds described herein (or a prodrug, pharmaceutically acceptable salt, or other pharmaceutically acceptable form thereof), and optionally a pharmaceutically acceptable excipient. In certain embodiments, these compositions optionally further comprise one or more additional therapeutic agents. Alternatively, a compound of the invention may be administered to a patient in need thereof in combination with the administration of one or more other therapeutic agents. For example, in the treatment of cancer, an additional therapeutic agents for conjoint administration or inclusion in a pharmaceutical composition with a compound of this invention may be an approved chemotherapeutic agent.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As described above, the pharmaceutical compositions of the present invention optionally comprise a pharmaceutically acceptable excipient, which, as used herein, includes any and all solvents, diluents, or other liquid vehicle, dispersion or suspension aids, surface active agents, isotonic agents, thickening or emulsifying agents, preservatives, antioxidants, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various excipients used in formulating pharmaceutical compositions and known techniques for the preparation thereof. Except insofar as any conventional excipient medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatine; talc; excipients such as cocoa butter and suppository waxes; oils such as peanut oil, cottonseed oil; safflower oil, sesame oil; olive oil; corn oil, and soybean oil; glycols; such as propylene glycol; esters such as ethyl oleate and ethyl laurate; agar, buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives, and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable media prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcelhdose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monosteamte, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols, and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols, and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a excipient system. Pharmaceutically effective excipients include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other excipient known in the art for topically administering pharmaceuticals. A more complete listing of art-known carvers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyarrisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum coreum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable excipient and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix (e.g., PLGA) or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent or anticancer agent), or they may achieve different effects (e.g., control of any adverse effects).

For example, other therapies or anticancer agents that may be used in combination with the inventive compounds of the present invention for cancer therapy include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferon, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, cyclophosphamide, melphalan, ifosfamide), antimetabolites (methotrexate), purine antagonists and pyrimidine antagonists (6-mercaptopurine, 5-fluorouracil, cytarabile, gemcitabine), spindle poisons (vinblastine, vincristine, vinorelbine, paclitaxel), podophyllotoxins (etoposide, irinotecan, topotecan), antibiotics (doxorubicin, bleomycin, mitomycin), nitrosoureas (carmustine, lomustine), inorganic ion (displatin, darboplatin), enzymes (asparaginase), and hormones (tamoxifen, leuprelide, flutamide, and megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/draglis&ame).

In certain embodiments, the pharmaceutical compositions of the present invention further comprise one or more additional therapeutically active ingredients (e.g., chemotherapeutic and/or palliative). For purposes of the invention, the term “palliative” refer, to treatment that is focused on the relief of symptoms of a disease and/or side effects of a therapeutic regimen, but is not curative. For example, palliative treatment encompasses painkillers, antinausea medication and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

Additionally, the present invention provides pharmaceutically acceptable derivatives of the inventive compounds, and methods of treating a subject using these compounds, pharmaceutical compositions thereof, or either of these in combination with one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a prodrug or other adduct or derivative of a compound of this invention which upon administration to a patient in need is capable of providing, directly or indirectly, a compound as otherwise described herein, or a metabolite or residue thereof.

Another aspect of the invention relates to a kit for conveniently and effectively carrying out the methods in accordance with the present invention. In general, the pharmaceutical pack or kit comprises one or more containers filled with one or more of the ingredients of the pharmaceutical compositions of the invention. Optionally associated with such container(s) can be a notice in the form prescribed by a governmental agency regulating the manufacture, use or sale of pharmaceutical products, which notice reflects approval by the agency of manufacture, use or sale for human administration.

### Pharmaceutical Uses and Methods of Treatment

In general, methods of using the compounds of the present invention comprise administering to a subject in need thereof a therapeutically effective amount of a compound of the present invention. Subjects suffering from a pathological condition responsive to chelation or sequestration of a trivalent metal can be treated with a therapeutically or prophylactically effective amount of an inventive compound, or pharmaceutical composition thereof. One particular type of pathological condition that is responsive to chelation of a trivalent metal is a trivalent metal overload condition (e.g., an iron overload condition or disease, an aluminum overload condition, a chromium overload condition). Another type of pathological condition that is responsive to metal chelation or sequestration is when the amount of free trivalent metal is elevated (e.g., in the serum or in a cell), such as when there is insufficient storage capacity for trivalent metals or an abnormality in the metal storage system that leads to metal release.

Iron overload conditions or diseases can be characterized by global iron overload or focal iron overload. Global iron overload conditions generally involve an excess of iron in multiple tissues or excess iron located throughout an organism. Global iron overload conditions can result from excess uptake of iron by a subject, excess storage and/or retention of iron, from, for example, dietary iron or blood transfusions. One global iron overload condition is primary hemochromatosis, which is typically a genetic disorder. A second global iron overload condition is secondary hemochromatosis, which is typically the result of receiving multiple (chronic) blood transfusions. Blood transfusions are often required for subjects suffering from thalassemia or sickle cell anemia. A type of dietary iron overload is referred to as Bantu siderosis, which is associated with the ingestion of homebrewed beer with high iron content.

In focal iron overload conditions, the excess iron is limited to one or a few cell types or tissues or a particular organ. Alternatively, symptoms associated with the excess iron are limited to a discrete organ, such as the heart, lungs, liver, pancreas, kidneys, or brain. It is believed that focal iron overload can lead to neurological or neurodegenerative disorders such as Parkinson's disease, Alzheimer's disease, Huntington's disease, neuroferritinopathy, amyotrophic lateral sclerosis, and multiple sclerosis. Pathological conditions that benefit from metal chelation or sequestration are often associated with deposition of the metal in the tissues of a subject. Deposition can occur globally or focally.

In humans with iron overload disease, the toxicity associated with an excess of this metal derives from iron's interaction with reactive oxygen species, for instance, endogenous hydrogen peroxide (H₂O₂).¹⁻⁴ In the presence of Fe(II), H₂O₂ is reduced to the hydroxyl radical (HO^{•}), a very reactive species, and HO⁻, the Fenton reaction. The hydroxyl radical reacts very quickly with a variety of cellular constituents and can initiate free radicals and radical-mediated chain processes that damage DNA and membranes, as well as produce carcinogens.^{2,5,6} The Fe(III) liberated can be reduced back to Fe(II) via a variety of biological reductants (e.g., ascorbate, glutathione), a problematic cycle.

The iron-mediated damage can be focal, as in reperfusion damage,⁷ Parkinson's,⁸ and Friedreich's ataxia,⁹ or global, as in transfusional iron overload, e.g., thalassemia,¹⁰ sickle cell disease,^{10,11} and myelodysplasia,¹² with multiple organ involvement. The solution in both scenarios is the same: chelate and promote the excretion of excess unmanaged iron.

While humans have a highly efficient iron management system in which they absorb and excrete about 1 mg of iron daily, there is no conduit for the excretion of excess metal. Transfusion-dependent anemias, like thalassemia, lead to a build up of iron in the liver, heart, pancreas, and elsewhere resulting in (i) liver disease that may progress to cirrhosis,¹³⁻¹⁵ (ii) diabetes related both to iron-induced decreases in pancreatic β-cell secretion and to increases in hepatic insulin resistance,^{16,17} and (iii) heart disease. Cardiac failure is still the leading cause of death in thalassemia major and related forms of transfusional iron overload.¹⁸⁻²⁰

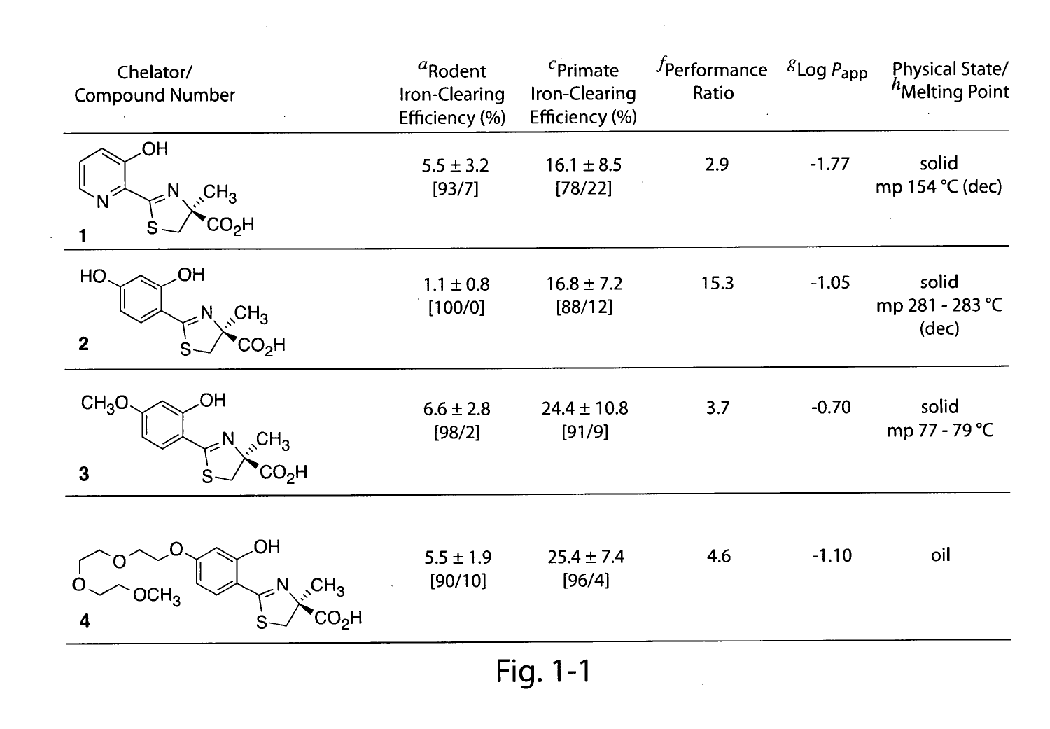

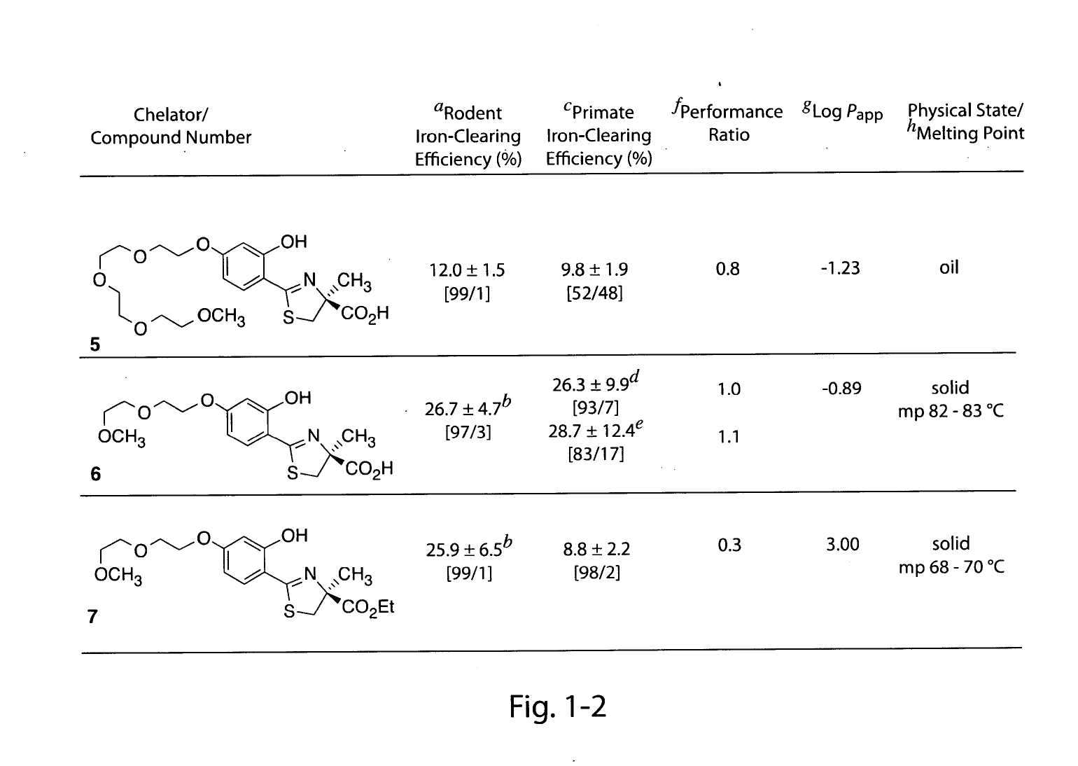

Treatment with a chelating agent capable of sequestering iron and permitting its excretion from the body is the only therapeutic approach available. Some of the iron-chelating agents that are now in use or that have been clinically evaluated include desferrioxamine B mesylate (DFO),²¹ 1,2-dimethyl-3-hydroxy-4-pyridinone (deferiprone, L1),²²⁻²⁵ 4-[3,5-bis(2-hydroxyphenyl)-1,2,4-triazol-1-yl]benzoic acid (deferasirox, ICL670A),²⁶⁻²⁹ and the desferrithiocin, (S)-4,5-dihydro-2-(3-hydroxy-2-pyridinyl)-4-methyl-4-thiazolecarboxylic acid (DFT, 1, FIG. 1) analogue, (S)-2-(2,4-dihydroxyphenyl)-4,5-dihydro-4-methyl-4-thiazolecarboxylic acid [deferitrin (2),³⁰ FIG. 1]. Each of these ligands presents with serious shortcomings. DFO must be given subcutaneously for protracted periods of time, e.g., 12 hours a day, five days a week, a serious patient compliance issue.³¹⁻³³ Deferiprone, while orally active, simply does not remove enough iron to maintain patients in a negative iron balance.²²⁻²⁵ Deferasirox did not show non-inferiority to DFO and is associated with numerous side effects; it has a very narrow therapeutic window.²⁶⁻²⁹ Finally, the clinical trial of 2 (FIG. 1) was abandoned by Genzyme because of renal toxicity.³⁰ However, deferitrin (2) has been reengineered, leading to the discovery that replacing the 4′-hydroxyl on the aromatic ring of 2 with a 3,6,9-trioxadecyloxy polyether group solved the renal toxicity issue;³⁴ iron clearing efficiency (ICE) was also improved. The boundary condition set by many hematologists is that the chelator should be able to remove 450 μg/kg/day of the metal.³⁵

A subject in need of oxidative stress reduction can have one or more of the following conditions: decreased levels of reducing agents, increased levels of reactive oxygen species, mutations in or decreased levels of antioxidant enzymes (e.g., Cu/Zn superoxide dismutase, Mn superoxide dismutase, glutathione reductase, glutathione peroxidase, thioredoxin, thioredoxin peroxidase, DT-diaphorase), mutations in or decreased levels of metal-binding proteins (e.g., transferrin, ferritin, ceruloplasmin, albumin, metallothionein), mutated or overactive enzymes capable of producing superoxide (e.g., nitric oxide synthase, NADPH oxidases, xanthine oxidase, NADH oxidase, aldehyde oxidase, dihydroorotate dehydrogenase, cytochrome c oxidase), and radiation injury. Increased or decreased levels of reducing agents, reactive oxygen species, and proteins are determined relative to the amount of such substances typically found in healthy persons. A subject in need of oxidative stress reduction can be suffering from an ischemic episode. Ischemic episodes can occur when there is mechanical obstruction of the blood supply, such as from arterial narrowing or disruption. Myocardial ischemia, which can give rise to angina pectoris and myocardial infarctions, results from inadequate circulation of blood to the myocardium, usually due to coronary artery disease. Ischemic episodes in the brain that resolve within 24 hours are referred to as transient ischemic attacks. A longer-lasting ischemic episode, a stroke, involves irreversible brain damage, where the type and severity of symptoms depend

on the location and extent of brain tissue whose access to blood circulation has been compromised. A subject at risk of suffering from an ischemic episode typically suffers from atherosclerosis, other disorders of the blood vessels, increased tendency of blood to clot, or heart disease. The compounds of the invention can be used to treat these disorders.

A subject in need of oxidative stress reduction can be suffering from inflammation. Inflammation is a fundamental pathologic process consisting of a complex of cytologic and chemical reactions that occur in blood vessels and adjacent tissues in response to an injury or abnormal stimulation caused by a physical, chemical, or biologic agent. Inflammatory disorders are characterized inflammation that lasts for an extended period (i.e., chronic inflammation) or that damages tissue. Such inflammatory disorders can affect a wide variety of tissues, such as respiratory tract, joints, bowels, and soft tissue. The compounds of the invention can be used to treat these disorders. Although not bound by theory, it is believed that the compounds of the invention derive their ability to reduce oxidative stress through various mechanisms. In one mechanism, the compound binds to a metal, particularly a redox-active metal (e.g., iron), and fills all of the coordination sites of the metal. When all of the metal coordination sites are filled, it is believed that oxidation and/or reducing agents have a diminished ability to interact with the metal and cause redox cycling. In another mechanism, the compound stabilizes the metal in a particular oxidation state, such that it is less likely to undergo redox cycling. In yet another mechanism, the compound itself has antioxidant activity (e.g., free radical scavenging, scavenging of reactive oxygen or nitrogen species). Desferrithiocin and its derivatives and analogues are known to have intrinsic antioxidant activity, as described in U.S. Application Publication No. 2004/0044220, published Mar. 4, 2004; U.S. Application Publication No. 2004/0132789, published Jul. 8, 2004; PCT Application No. WO2004/017959, published Mar. 4, 2004, U.S. Application Publication No. 2003/0236417, published Dec. 25, 2003; and U.S. Pat. Nos. 6,083,966, 6,559,315, 6,525,080, and 6,521,652 the contents of each of which are incorporated herein by reference.

Imaging or examining one or more organs, tissues, tumors, or a combination thereof can be conducted after a metal salt of a compound of the invention is administered to a subject. The methods of imaging and examining are intended to encompass various instrumental techniques used for diagnosis, such as x-ray methods (including CT scans and conventional x-ray images), magnetic imaging (magnetic resonance imaging, electron paramagnetic resonance imaging) and radiochemical methods. Typically, the metal salts used in imaging or examining serve as a contrast agent. Therefore in one embodiment the metal complexes or metal salts of compounds of the present invention can be used as contrast agents for example in imaging or examining one or more organs, for example, the gastrointestinal tract. Metals that can serve as contrast agents include gadolinium, iron, manganese, chromium, dysprosium, technetium, scandium, barium, aluminum and holmium, preferably as trications. Radioactive metal salts can be made from isotopes including ²⁴¹Am, ⁵¹Cr, ⁶⁰Co, ⁵⁷Co, ⁵⁸Co, ⁶⁴Cu, ¹⁵³Gd, ⁶⁷Ga, ¹⁹⁸Au, ^{113m}In, ¹¹¹In, ⁵⁹Fe, ⁵⁵Fe, ¹⁹⁷Hg, ²⁰³Hg, ^{99m}Tc, ²⁰¹Tl, and ¹⁶⁹Yb, again preferably when the metal is present as a trivalent cation.

Neoplastic disease is characterized by an abnormal tissue that grows by cellular proliferation more rapidly than normal tissue. The abnormal tissue continues to grow after the stimuli that initiated the new growth cease. Neoplasms show a partial or complete lack of structural organization and functional coordination with the normal tissue, and usually form a distinct mass of tissue that may be either benign, or malignant. Neoplasms can occur, for example, in a wide variety of tissues including brain, skin, mouth, nose, esophagus, lungs, stomach, pancreas, liver, bladder, ovary, uterus, testicles, colon, and bone, as well as the immune system (lymph nodes) and endocrine system (thyroid gland, parathyroid glands, adrenal gland, thymus, pituitary gland, pineal gland). The compounds of this invention can be used to treat these disorders. Examples of tumors or cancers that can be treated by the invention include, but are not limited to, leukemia, Hodgkin's disease, non-Hodgkin's lymphomas, multiple myeloma, macroglobulinemia, polycythemia vera, lung tumors, head and neck tumors, brain tumors (neuroblastoma), endometrial tumors, ovarian tumors, cervical tumors, breast tumors, choriocarcinoma, testical tumors, prostate tumor, Wilms' tumor, thyroid tumors, adrenal tumors, stomach tumor, pancreal tumors, colonic tumors, carcinoids, insulinoma, bone tumors (osteogenic sarcoma), miscellaneous sarcomas and skin cancer (melanoma).

A preneoplastic condition precedes the formation of a benign or malignant neoplasm. A precancerous lesion typically forms before a malignant neoplasm. Preneoplasms include photodermatitis, x-ray dermatitis, tar dermatitis, arsenic dermatitis, lupus dermatitis, senile keratosis, Paget disease, condylomata, burn scar, syphilitic scar, fistula scar, ulcus cruris scar, chronic ulcer, varicose ulcer, bone fistula, rectal fistula, Barrett esophagus, gastric ulcer, gastritis, cholelithiasis, kraurosis vulvae, nevus pigmentosus, Bowen dermatosis, xeroderma pigmentosum, erythroplasia, leukoplakia, Paget disease of bone, exostoses, ecchondroma, osteitis fibrosa, leontiasis ossea, neurofibromatosis, polyposis, hydatidiform mole, adenomatous hyperplasia, and struma nodosa. The compounds of this invention can be used to treat these disorders.

A “subject” is typically a human, but can also be an animal in need of treatment, e.g., companion animals (e.g., dogs, cats, and the like), farm animals (e.g., cows, pigs, horses, sheep, goats and the like) and laboratory animals (e.g., rats, mice, guinea pigs, non-human primates and the like).

The compounds and pharmaceutical compositions of the present invention can be administered by an appropriate route. Suitable routes of administration include, but are not limited to, orally, intraperitoneally, subcutaneously, intramuscularly, transdermally, rectally, sublingualis intravenously, buccally, or inhalationally. Preferably, compounds and pharmaceutical compositions of the invention are administered orally. The pharmaceutical compositions of the invention preferably contain a pharmaceutically acceptable excipient suitable for rendering the compound or mixture administrable orally, parenterally, intravenously, intradermally, intramuscularly or subcutaneously, rectally, via inhalation or via buccal administration, or transdermally. The active ingredients may be admixed or compounded with a conventional, pharmaceutically acceptable excipient. It will be understood by those skilled in the art that a mode of administration, vehicle, excipient or carrier conventionally employed and which is inert with respect to the active agent may be utilized for preparing and administering the pharmaceutical compositions of the present invention. Illustrative of such methods, vehicles, excipients, and carriers are those described, for example, in Remington's Pharmaceutical Sciences, 18th ed. (1990), the disclosure of which is incorporated herein by reference. The formulations of the present invention for use in a subject comprise the agent, together with one or more acceptable excipient thereof, and optionally other therapeutic agents. The excipient must be “acceptable” in the sense of being compatible with the other ingredients of the formulation and not deleterious to the recipient thereof. The formulations can conveniently be presented in unit dosage form and can be prepared by any of the methods well known in the art of pharmacy. All methods include the step of bringing into association the agent with the excipient which constitutes one or more accessory ingredients. In general, the formulations are prepared by uniformly and intimately bringing into association the agent with the excipient and then, if necessary, dividing the product into unit dosages thereof.

Forms suitable for oral administration include tablets, troches, capsules, elixirs, suspensions, syrups, wafers, chewing gum, or the like prepared by art recognized procedures. The amount of active compound in such therapeutically useful compositions or preparations is such that a suitable dosage will be obtained. A syrup formulation will generally consist of a suspension or solution of the compound or salt in a liquid carrier, for example, ethanol, glycerine or water, with a flavoring or coloring agent. Where the composition is in the form of a tablet, one or more pharmaceutical excipient routinely used for preparing solid formulations can be employed. Examples of such excipient include magnesium stearate, starch, lactose and sucrose. Where the composition is in the form of a capsule, the use of routine encapsulation is generally suitable, for example, using the aforementioned excipient in a hard gelatin capsule shell. Where the composition is in the form of a soft gelatin shell capsule, pharmaceutical excipient routinely used for preparing dispersions or suspensions can be considered, for example, aqueous gums, celluloses, silicates, or oils, and are incorporated in a soft gelatin capsule shell.

Formulations suitable for parenteral administration conveniently include sterile aqueous preparations of the agents that are preferably isotonic with the blood of the recipient. Suitable excipient solutions include phosphate buffered saline, saline, water, lactated Ringer's or dextrose (5% in water). Such formulations can be conveniently prepared by admixing the agent with water to produce a solution or suspension, which is filled into a sterile container and sealed against bacterial contamination. Preferably, sterile materials are used under aseptic manufacturing conditions to avoid the need for terminal sterilization. Such formulations can optionally contain one or more additional ingredients, which can include preservatives such as methyl hydroxybenzoate, chlorocresol, metacresol, phenol and benzalkonium chloride. Such materials are of special value when the formulations are presented in multidose containers.

Buffers can also be included to provide a suitable pH value for the formulation. Suitable buffer materials include sodium phosphate and acetate. Sodium chloride or glycerin can be used to render a formulation isotonic with the blood.

If desired, a formulation can be filled into containers under an inert atmosphere such as nitrogen and can be conveniently presented in unit dose or multi-dose form, for example, in a sealed ampoule.

Those skilled in the art will be aware that the amounts of the various components of the compositions of the invention to be administered in accordance with the method of the invention to a subject will depend upon those factors noted above.

A typical suppository formulation includes the compound or a pharmaceutically acceptable salt thereof which is active when administered in this way, with a binding and/or lubricating agent, for example, polymeric glycols, gelatins, cocoa-butter, or other low melting vegetable waxes or fats. Typical transdermal formulations include a conventional aqueous or nonaqueous vehicle, for example, a cream, ointment; lotion, or paste or are in the form of a medicated plastic, patch or membrane.

Typical compositions for inhalation are in the form of a solution, suspension, or emulsion that can be administered in the form of an aerosol using a conventional propellant such as dichlorodifluoromethane or trichlorofluoromethane.

The therapeutically effective amount of a compound or pharmaceutical composition of the invention depends, in each case, upon several factors, e.g., the health, age, gender, size, and condition of the subject to be treated, the intended mode of administration, and the capacity of the subject to incorporate the intended dosage form, among others. A therapeutically effective amount of an active agent is an amount sufficient to have the desired effect for the condition being treated. For example, in a method of treating of a neoplastic or a preneoplastic condition, the desired effect is partial or total inhibition, delay or prevention of the progression of cancer or the tumor including cancer metastasis; inhibition, delay or prevention of the recurrence of cancer or the tumor including cancer metastasis; or the prevention of the onset or development of cancer or a tumor (chemoprevention) in a mammal, for example a human. In a method of treating a subject with a condition treatable by chelating or sequestering a metal ion, a therapeutically effective amount of an active agent is, for example, an amount sufficient to reduce the burden of the metal in the subject, reduce the symptoms associated with the metal ion or prevent, inhibit or delay the onset and/or severity of symptoms associated with the presence of the metal. In a method of reducing oxidative stress in a subject in need of treatment thereof, a therapeutically effective amount of an active agent is, for example, an amount sufficient to reduce symptoms associated with oxidative stress or prevent, inhibit or delay the onset and/or severity of symptoms associated with oxidative stress.

A typical total daily dose of a compound of the invention to be administered to a subject (assuming an average 70 kg subject) is from approximately 5 mg to approximately 10,000 mg, (for example 0.07 mg/kg to 143 mg/kg), and preferably from approximately 50 mg to approximately 5,000 mg approximately 100 mg to approximately 2,000 mg approximately 300 mg to approximately 1,000 mg. For iron overload therapy, a daily dose of a compound of the invention should remove a minimum of from approximately 0.25 to approximately 0.40 mg of iron per kilogram of body mass per day. The dosage can be administered orally in several, for example, one, two, three, four, six, eight, twelve, or more individual doses.

### Preparation of Compounds of the Invention

Compounds of formula (Ia) can be synthesized, for example, by reacting a polyethylene glycol chain of formula:

X—O—[(CH₂)ₙ—O]ₓ—R

wherein X is a leaving group;

with an alcohol of formula (III):

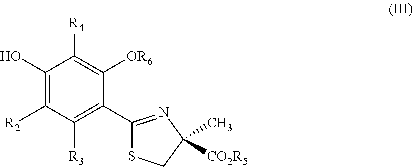

under suitable conditions to yield a compound of formula (Ia).

As would be appreciated by one of skill in the art, the suitable reaction conditions include, temperature, solvent, reaction time, concentration, etc.

In certain embodiments, the polyethylene glycol chain and alcohol can be reacted under basic conditions. In other embodiments, the polyethylene glycol chain and alcohol can be reacted in an alkaline solution. In certain embodiments, the polyethylene glycol chain and alcohol can be reacted in the presence of a base. In other embodiments, the base is an alkali. In further embodiments, the base is a basic salt. In still further embodiments, the basic salt is sodium hydroxide, potassium hydroxide, barium hydroxide, cesium hydroxide, calcium hydroxide, lithium hydroxide, or magnesium hydroxide. In certain embodiments, the basic salt is calcium carbonate, or potassium carbonate.

In certain embodiments, the base is an alkoxide. In still further embodiments, the alkoxide is an alkoxide salt. In certain embodiments, the alkoxide is sodium ethoxide, sodium methoxide, aluminum isopropoxide, or potassium tert-butoxide.

In certain embodiments, the solvent is a polar solvent. In other embodiments, the solvent is a non-nucleophilic solvent. In still other embodiments, the solvent is a polar aprotic solvent. In further embodiments, the solvent is DMF, dioxane, HMPT (hexamethylphosphorotriamide), THF, or Et₂O. In a certain embodiments, the solvent is acetone.

In certain embodiments, the polyethylene glycol chain is in a solution of 0.01-0.5 M. In other embodiments, the polyethylene glycol chain is in solution of 0.1-0.25 M. In other embodiments, the polyethylene glycol chain is in a solution of 0.15 M. In a specific embodiment, the polyethylene glycol chain is in acetone at a concentration of 0.15 M.

In another aspect of the invention, a method for obtaining compound of general formula (Ia) as a solid is provided.

In certain embodiments, the method for obtaining a compound of formula (Ia) further comprises the step of crystallization. In certain embodiments, the crystallization is a direct crystallization. In other embodiments, the crystallization is a recrystallization. In certain embodiments, the recrystallization is a single-solvent recrystallization. In other embodiments, the recrystallization is a multi-solvent recrystallization. In further embodiments, the recrystallization is a hot filtration recrystallization. In certain embodiments, the crystallization is spontaneous. In other embodiments, the crystallization requires seeding. In further embodiments, the crystallization is a trituration.

In certain embodiments, the crystallization solvent is a polar aprotic solvent. In other embodiments, the polar aprotic solvent is EtOAc. In other embodiments, the crystallization solvent is a non-polar solvent. In certain embodiments, the crystallization solvent is hexane. In certain embodiments, the crystallization solvents are a polar aprotic solvent and a non-polar solvent. In other example the crystallization solvents are EtOAc and hexane.

In certain embodiments, the ester of general formula (Ia) is synthesized as illustrated in Scheme 1.

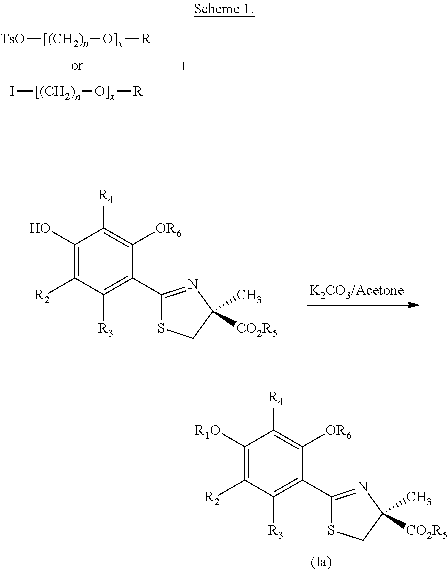

Compounds of formula (Ib) can be synthesized, for example, by ester hydrolysis of a compound of general formula (Ia).

In certain embodiments, the hydrolysis is an acid-catalyzed hydrolysis. In other embodiments, the hydrolysis is a base hydrolysis. In further embodiments, the base is an organic base. In certain embodiments, the base is an hydroxide. In other embodiments, the hydroxide is sodium hydroxide, potassium hydroxide, or calcium hydroxide. In further embodiment, the base is 1N NaOH.

In certain embodiments, the hydrolysis is carried out in a polar solvent. In other embodiments, the polar solvent is an alcohol. In further embodiments, the alcohol is primary alcohol. In other embodiments, the alcohol is a secondary alcohol. In certain embodiments, the alcohol is a tertiary alcohol. In other embodiments, the alcohol is methanol, ethanol, iso-propanol, n-butanol, iso-butanol, or tert-butanol.

In certain embodiments, the ester of general formula (Ib) is in a solution of 0.01-0.5 M. In other embodiments, the ester is in solution of 0.1-0.25 M. In other embodiments, the ester is in a solution of 0.1 M. In a specific embodiment, the ester is in methanol at a concentration of 0.1 M.

In certain embodiments, the method further comprises the step of acidification. In other embodiments, the acidification is performed with a monoprotic acid. In other embodiments, the acidification is performed with a polyprotic acid. In further embodiments, the acid is a mineral acid. In certain embodiments, the acid is an organic acid. In other embodiments, the acid is HCl.

In certain embodiments, the method for obtaining a compound of general formula (Ib) further comprises the step of crystallization. In certain embodiments, the crystallization is a direct crystallization. In other embodiments, the crystallization is a recrystallization. In certain embodiments, the recrystallization is a single-solvent recrystallization. In other embodiments, the recrystallization is a multi-solvent recrystallization. In further embodiments, the recrystallization is a hot filtration recrystallization. In certain embodiments, the crystallization is spontaneous. In other embodiments, the crystallization requires seeding. In further embodiments, the crystallization is a trituration.

In certain embodiments, the crystallization solvent is a polar aprotic solvent. In other embodiments, the polar aprotic solvent is EtOAc. In other embodiments, the crystallization solvent is a non-polar solvent. In certain embodiments, the crystallization solvent is hexane. In certain embodiments, the crystallization solvents are a polar aprotic solvent and a non-polar solvent. In other example the crystallization solvents are EtOAc and hexane.

In certain embodiments, the acid of general formula (Ib) is synthesized as illustrated in Scheme 2.

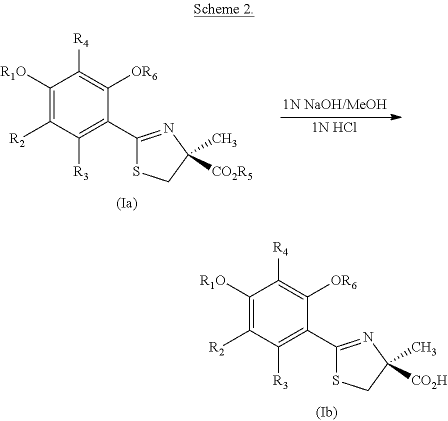

In certain embodiments, compounds of the invention are synthesized as illustrated in Scheme 3.

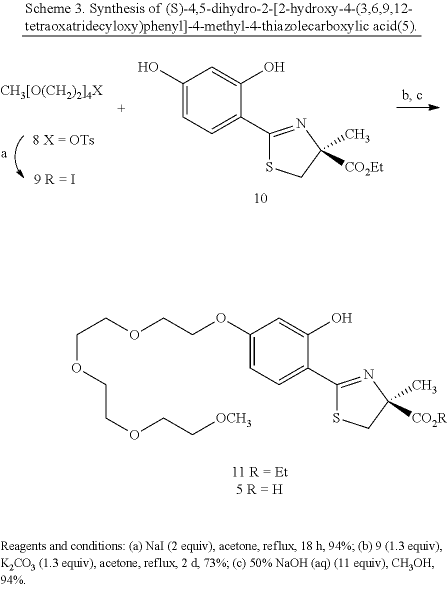

In certain embodiments, compounds of the invention are synthesized as illustrated in Scheme 4.

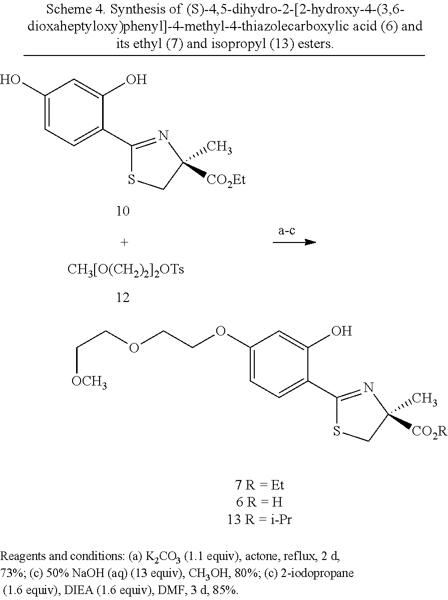

In certain embodiments, the methods described above are carried out in solution phase. In certain other embodiments, the methods described above are carried out on a solid phase. In certain embodiments, the synthetic method is amenable to high-throughput techniques or to techniques commonly used in combinatorial chemistry.

In certain embodiments, the starting material are synthesized. In other embodiments, the starting materials are purchased from a commercial source. The starting materials may be protected before reacting them.

In certain embodiments, the reaction mixture of the polyethylene glycol chain and the alchohl is heated. In other embodiments, the reaction temperature is 50-120° C. In yet other embodiments, the reaction temperature is 50-60° C. In still other embodiments, the reaction temperature is 60-70° C. In certain embodiments, the reaction temperature is 70-80° C. In other embodiments, the reaction temperature is 80-90° C. In yet other embodiments, the reaction temperature is 90-100° C. In still other embodiments, the reaction temperature is 100-110° C. In certain embodiments, the reaction temperature is 110-120° C. In a specific embodiment, the reaction temperature is 60° C.

### EXAMPLES

DFT (1) is a natural product iron chelator, a siderophore. It forms a tight 2:1 complex with Fe(III), has a log β₂ of 29.6,³⁶⁻³⁸ and was one of the first iron chelators shown to be orally active. It performed well in both the bile duct-cannulated rodent model (ICE, 5.5%)³⁹ and in the iron-overloaded C. apella primate (ICE, 16%). ^{40,41} Unfortunately, 1 was severely nephrotoxic.⁴¹ Nevertheless, the outstanding oral activity spurred a structure-activity study to identify an orally active and safe DFT analogue. The first goal was to define the minimal structural platform, pharmacophore, compatible with iron clearance upon oral administration.⁴²⁻⁴⁴

Removal of the pyridine nitrogen of DFT provided (S)-4,5-dihydro-2-(2-hydroxyphenyl)-4-methyl-4-thiazolecarboxylic acid [(S)-DADFT],⁴⁴ the parent ligand of the desaza (DA) series. Substitution of the 4-methyl of (S)-DADFT with a hydrogen led to (S)-4,5-dihydro-2-(2-hydroxyphenyl)-4-thiazolecarboxylic acid [(S)-DADMDFT],^{41,44} the platform for the ensuing DADM systems. In the course of additional structure activity relationship (SAR) studies, we were able to determine that within a given family of ligands, e.g., the DADFTs or the DADMDFTs, that the chelator's log Pₐₚₚ, lipophilicity, had a profound effect on both ICE and toxicity.^{34,43,45} In each family, as the lipophilicity decreases, i.e., the log Pₐₚₚ becomes more negative, the toxicity also decreases. The more lipophilic chelators generally had greater ICE and increased toxicity.^{34,43,45} It is critical to remain within families when making these comparisons. For example, there is no relationship between the log Pₐₚₚ, ICE, and toxicity of DFT itself versus the log Pₐₚₚ, ICE, and toxicity of its analogues. However, in the case of the desaza family of ligands, for example, when a 4′-(CH₃O) group was fixed in place of the 4′-(HO) of 2, providing (S)-4,5-dihydro-2-(2-hydroxy-4-methoxyphenyl)-4-methyl-4-thiazolecarboxylic acid (3, FIG. 1), the molecule's lipophilicity increased, as did its ICE and toxicity.^{34,43} This ligand is very lipophilic, log Pₐₚₚ=−0.70, and a very effective iron chelator when given orally to rodents³⁴ or primates⁴³ (FIG. 1). Unfortunately, the ligand was also very nephrotoxic.³⁴ The question then became how to balance the lipophilicity/toxicity interaction while iron-clearing efficiency is maintained.

Ultimately, we discovered that fixing a polyether moiety, a 3,6,9-trioxadecyloxy group, to the 4′-position of 2, providing (S)-4,5-dihydro-2-[2-hydroxy-4-(3,6,9-trioxadecyloxy)phenyl]-4-methyl-4-thiazolecarboxylic acid (4, FIG. 1), resulted in a ligand that retained the ICE properties of 3, but was much less lipophilic and less toxic than 3.³⁴ This polyether fragment has been fixed to one of three positions on the aromatic ring, 3′-, 4′-, or 5′-.^{34,46} The iron-clearing efficiency in rodents and primates is shown to be very sensitive to which positional isomer is evaluated.^{34,46} In rodents, the polyethers had uniformly higher ICEs than their corresponding parent ligands. There was also a profound reduction in toxicity, particularly renal toxicity.^{34,46,47} In the primate model, the ICEs for both the 3′- and 4′-polyethers were similar to the corresponding phenolic parent, e.g., the 3′-(HO) isomer of deferitrin (2) and 2, respectively.⁴⁶ However, the ICE of the 5′-polyether substituted ligand decreased relative to its parent.⁴⁶ What remained unclear was the quantitative significance of the length of the polyether backbone on the properties of the ligands, the subject of this work.

In the current study, additional polyether analogues of 2 were synthesized (FIG. 1). Specifically, the 3,6,9-trioxadecyloxy substituent at the 4′-position of ligand 4 was both lengthened to provide (S)-4,5-dihydro-2-[2-hydroxy-4-(3,6,9,12-tetraoxamidecyloxy)phenyl]-4-methyl-4-thiazolecarboxylic acid (5), and shortened to provide (S)-4,5-dihydro-2-[2-hydroxy-4-(3,6-dioxaheptyloxy)phenyl]-4-methyl-4-thiazolecarboxylic acid (6). The ethyl ester of 6, ethyl (S)-4,5-dihydro-2-[2-hydroxy-4-(3,6-dioxaheptyloxy)phenyl]-4-methyl-4-thiazolecarboxylate (7), was also prepared. Three questions were addressed regarding the structural changes in ligand 2:1) the effect on lipophilicity, 2) the effect on the iron clearing efficiency in the bile duct-cannulated rodent and primate models, and 3) the effect on the physiochemical properties of the ligand. We have consistently seen that, within a given family, ligands with greater lipophilicity are more efficient iron chelators, but are also more toxic,^{34,43,45} thus issues 1 and 2. We have also observed that the polyether acids for the 3′- and 4′-3,6,9-trioxadecyloxy analogues are oils, and in most cases, the salts are hygroscopic. A crystalline solid ligand would offer greater flexibility in dosage forms.

Deferitrin (2) was converted to ethyl (S)-2-(2,4-dihydroxyphenyl)-4,5-dihydro-4-methyl-4-thiazolecarboxylate (10)⁴⁸ in this laboratory. With the carboxylate group protected as an ester, alkylation of the less sterically hindered 4′-hydroxy of 10 in the presence of the 2′-hydroxy, an iron chelating site, has generated numerous desferrithiocin analogues, including 3-6 (FIG. 1).^{34,43}

Thus, O-monoalkylation of ethyl ester 10 with 13-iodo-2,5,8,11-tetraoxamidecane (9) using potassium carbonate in refluxing acetone generated masked chelator 11 in 73% yield (Scheme 3). Tetraether iodide 9 was readily accessed in 94% yield from tosylate 8,^{49,50} employing sodium iodide (2 equiv) in refluxing acetone, as alkylating agent 8 possesses similar chromatographic properties to ester 11. Removal of the ester-protecting group of 11 in base completed the synthesis of 3,6,9,12-tetraoxamidecyloxy ligand 5, a homologue of 4⁴⁷, with an additional ethyleneoxy unit in the polyether chain, in 94% yield.

The synthesis of the 3,6-dioxaheptyloxy ligand (6), the analogue of chelator 4 with one less ethyleneoxy unit in the polyether chain, was prepared using similar strategy (Scheme 4). 4′-O-Alkylation of ethyl ester 10 with 3,6-dioxaheptyl 4-toluenesulfonate (12)⁴⁹ generated 7 in 73% recrystallized yield. Unmasking ester 7 under alkaline conditions furnished the shorter 4′-polyether-derived iron chelator 6 in 80% recrystallized yield. Both ligand 6 and its ethyl ester 7 are crystalline solids, and thus offer clear advantages both in large scale synthesis and in dosage forms over previously reported polyether-substituted DFTs, which are oils.^{34,46,47} Carboxylic acid 6 was esterified using 2-iodopropane and N,N-diisopropylethylamine (DIEA) (1.6 equiv each) in DMF, providing isopropyl (S)-4,5-dihydro-2-[2-hydroxy-4-(3,6-dioxaheptyloxy)phenyl]-4-methyl-4-thiazolecarboxylate (13) in 85% yield as an oil (Scheme 4). This is consistent with the idea that the structural boundary conditions for ligand crystallinity are very narrow.

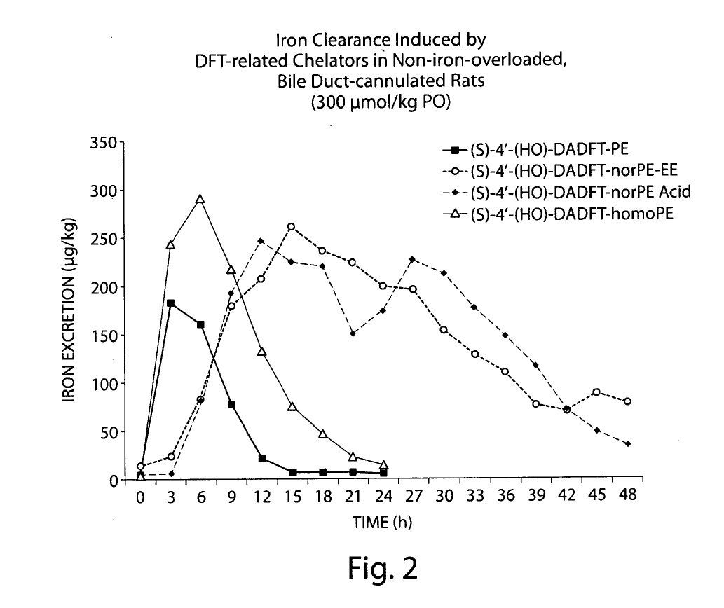

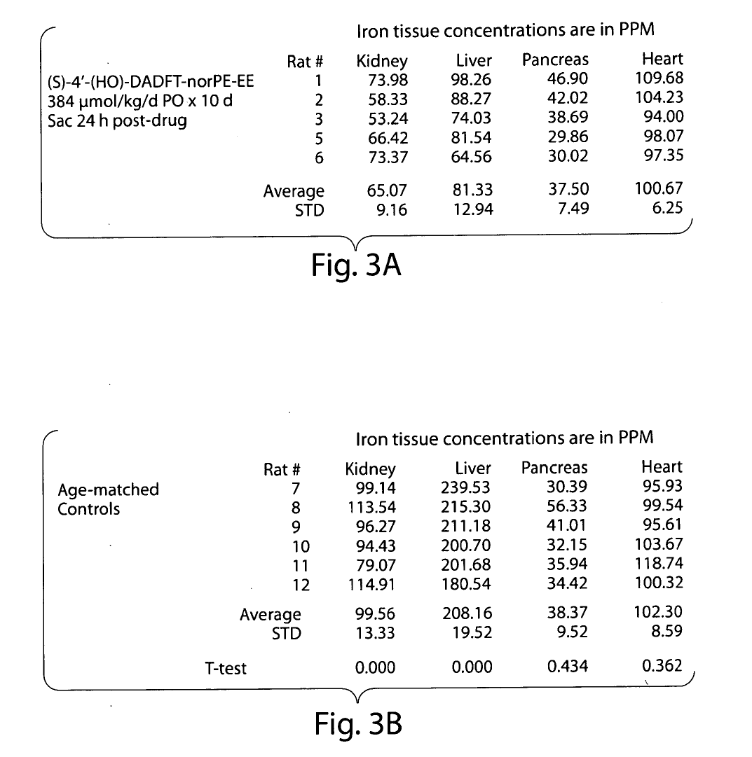

Single crystal X-ray analysis confirmed that chelator 6 (FIG. 1) and its ethyl ester 7 (FIG. 2) exist in the (S)-configuration. Both 6 and 7 crystallize in the monoclinic lattices, space group P2₁, with two molecules in the unit cell. Moreover, acid 6 has unit cell dimensions of a=5.5157(5) Å, b=8.8988(8) Å, and c=17.3671(16) Å with α and γ=90° and β=98.322(1)°. The unit cell dimensions of ester 7 are a=7.7798(6) Å, b=8.9780(6) Å, and c=14.1119(10) Å, also with α and γ=90° but β=106.078(1)°. Unit cell volumes (Å³) of 6 and 7 are 843.46(13) and 947.12(12), respectively. In the crystal lattice of 6, the acidic hydrogen is bonded to O6A of the carboxylate group, resulting in a neutral molecule (FIG. 1). However, parent ligand 2 (FIG. 1) with a strongly electron donating 4′-hydroxy is zwitterionic, that is, an iminium ion is observed by X-ray crystallography.⁵¹ Thus, not unexpectedly, deferitrin (2) (log Pₐₚₚ=−1.05) is more hydrophilic than polyether chelator 6 (log Pₐₚₚ=−0.89).

The partition values between octanol and water (at pH 7.4, Tris buffer) were determined using a “shake flask” direct method of measuring log Pₐₚₚ values.⁵² The fraction of drug in the octanol is then expressed as log Pₐₚₚ. These values varied widely (FIG. 1), from log Pₐₚₚ=−1.77 for 1 to log Pₐₚₚ−3.00 for 7. This represents a greater than 58.000-fold difference in partition. The most lipophilic chelator, 7, is 11,220 times more lipophilic than the parent 2.

Animal models: There are no dependable in vitro assays for predicting the in vivo efficacy of an iron decorporation agent.^{53,54} While tight iron binding is a necessary requirement for an effective iron chelator, it is not sufficient.⁵⁵ Once having established that a ligand platform, pharmacophore, binds iron tightly, e.g., desferrithiocin,^{37,38} Structure-activity relationship studies focused on minimizing toxicity while optimizing iron clearance are carried out.

Chelator-induced iron clearance in non-iron-overloaded, bile duct-cannulated rodents: As used herein, “iron-clearing efficiency” (ICE) is used as a measure of the amount of iron excretion induced by a chelator. The ICE, expressed as a percent, is calculated as (ligand-induced iron excretion/theoretical iron excretion)×100. To illustrate, the theoretical iron excretion after administration of one millimole of DFO, a hexadentate chelator that forms a 1:1 complex with Fe(III), is one milli-g-atom of iron. Two millimoles of desferrithiocin (DFT, 1, FIG. 1), a tridentate chelator which forms a 2:1 complex with Fe(III), are required for the theoretical excretion of one milli-g-atom of iron. In the rodents, in each instance, the polyether analogues are better iron clearing agents than their phenolic counterparts, e.g., 2 vs. 4, 5, 6, or 7 (FIG. 1). Historical data (compounds 1-4)^{34,39,43} has been included for comparative purposes. The ICE of the 3,6,9-trioxadecyloxy analogue (4) is five times greater than that of the parent ligand (2), 5.5±1.9% vs 1.1±0.8% (p<0.003), respectively.³⁴ The longer ether analogue, 3,6,9,12-tetraoxamidecyloxy analogue (5), is nearly 11 times as efficient as 2, with an ICE of 12.0±1.5% (p<0.001). The shorter ether analogue, the 3,6-dioxaheptoxy ligand (6), and its corresponding ethyl ester (7) are highly crystalline solids that were administered to the rats in capsules.⁵⁶ Both ligands are approximately 24 times as effective as the parent compound 2, with ICE values of 26.7±4.7% (p<0.001) and 25.9±6.5% (p<0.001), respectively. The difference in iron clearing properties between 4 and 5 versus 6 and 7 is likely due to the differences in lipophilicity as reflected in the log Pₐₚₚ (FIG. 1). This observation has remained remarkably consistent throughout our studies with DFT analogues.^{34,43,45} The latter two ligands are more lipophilic, with larger log Pₐₚₚ values.

The biliary ferrokinetics profiles of the ligands, 2 and 4-7, are very different (FIG. 2) and clearly related to differences in the polyether backbones. The maximum iron clearance (MIC) of the parent drug, deferitrin (2), occurs at 3 h, with iron clearance virtually over at 9 h. The trioxa polyether (4) also has an MIC at 3 h, with iron excretion extending out to 12 h. The tetraoxa ether analogue 5 has an MIC at 6 h; iron excretion continues for 24 h. The MIC of the dioxa ether analogue 6 and its corresponding ester 7 do not occur until 12-15 h, and iron excretion had not returned to baseline levels even 48 h post-drug. Note that although the biliary ferrokinetics curve of 6 may appear to be biphasic (FIG. 2), the reason for this unusual line shape is that several animals had temporarily obstructed bile flow. While the concentration of iron in the bile remained the same, the bile volume, and thus overall iron excretion, decreased. Once the obstruction was resolved, bile volume and overall iron excretion normalized.

Chelator-induced iron clearance in iron-overloaded primates: The iron clearance data for the chelators in the primates are described in FIG. 1. Historical data (compounds 1-4) has been included for comparative purposes.^{34,39,40,42,43} Ligand 2 had an ICE of 16.8±7.2%,³⁴ while the ICE of 4 is 25.4±7.4%.³⁴ The ICE of the longer 3,6,9,12-tetraoxa analogue (5) was significantly less, 9.8±1.9% (p<0.001). The shorter 3,6-dioxa analogue, 6, had an ICE of 26.3±9.9% when it was given to the primates in capsules; the ICE was virtually identical when it administered by gavage as its sodium salt, 28.7±12.4% (p>0.05). The similarity in ICE of 6 between the encapsulated acid and the sodium salt given by gavage suggest comparable pharmacokinetics. The ester of ligand 6, compound 7, performed relatively poorly in the primates, with an ICE of only 8.8±2.2%.

There are some notable differences between the current ICE data and previously reported studies.^{34,43,46} In the past, ligands generally performed significantly better in the iron-overloaded primates than in the non-iron-overloaded rodents. For example, we reported that the performance ratio (PR), defined as the mean ICEₚᵣᵢₘₐₜₑₛ/ICE_{rodents}, of analogues 2-4 are 15.3, 3.7, and 4.6, respectively (FIG. 1).⁴⁶ In the current study, the PR of ligand 5 is 0.82, while that of 6 is 1.0. Previously, the only ligand that behaved so alike in primates and rodents was the 5′-isomer of 4, which also had a performance ratio of 1.⁴⁶ However, on an absolute basis, the ICE for this chelator in primates (8.1±2.8%) was, in fact, poor. In current study, ligand 6 performed exceptionally well in both rodents and primates (ICE>26%), suggesting a higher index of success in humans. The ester of 6, ligand 7, on the other hand, had a very low performance ratio (0.33), lower than we have previously observed.

The profound difference between the ICE of the parent acid chelator 6 versus that of the ester 7 in rodents and primates is consistent with two possible explanations: 1) The ester is poorly absorbed from the gastrointestinal (GI) tract in the primates, or 2) The primate non-specific serum esterases simply may not cleave ester 7 to the active chelator acid 6. An experiment was performed using rat and monkey plasma in an attempt to determine if the relatively poor ICE of 7 in the primates was due to interspecies differences in hydrolysis. When 7 was solubilized in DMSO and incubated at 37° C. with rat plasma, all of the ester had been converted to the active acid 6 within 1-2 h. This was also the case when the experiment was carried out with plasma from Cebus apella monkeys. Thus, there is no difference in the hydrolysis of 7 between the rats and the primates. Therefore, the poor ICE of 7 in the monkeys is consistent with the idea that the ester is absorbed much more effectively from the GI tract of the rodents than from the GI tract of the primates. Control experiments were also performed in which saline was used in place of the rat or monkey plasma. Note that when 7 was solubilized in DMSO and incubated with saline in place of the rat or monkey plasma, all of the drug remained in the form of the ester.

Toxicity profile of (S)-4,5-dihydro-2-[2-hydroxy-4-(3,6-dioxaheptyloxy)phenyl]-4-methyl-4-thiazolecarboxylic acid (6) and its ethyl ester (7). Ten-day toxicity trials have been carried out in rats on both ligands 6 and 7. The drugs were given to the animals orally once daily at a dose of 384 μmmol/kg/d (equivalent to 100 mg/kg of DFT sodium salt). Additional age-matched animals served as untreated controls. The animals were euthanized on day 11, one day after the last dose of drug. Extensive tissues were sent out for histopathological examination. The kidney, liver, pancreas, and heart of test and control animals were removed and wet-ashed to assess their iron content.

Because ligand 6 was such an effective iron chelator in both the rats and the primates, its toxicity profile is most relevant. The key comment from the pathologist was that “The tissues from rats in Group 1 [test group] cannot be reliably differentiated histologically from the tissues from rats in Group 2 [control animals].” This was very encouraging, especially in view of how much iron the chelator removed from the liver and heart in such a short period of time. However, in spite of this outcome, it is clear that any protracted toxicity trials in rodents will have to include groups of both iron-loaded and non-iron-loaded animals, as a 28-day exposure to 6 could reduce the liver iron stores sufficiently to lead to toxicity.

The scenario with the ethyl ester of 6, compound 7, was somewhat different. While its ICE was excellent in rodents, along with an impressive reduction in liver and renal iron content, ester 7 did present with some renal toxicity. Mild to moderate vacuolar degeneration of the proximal tubular epithelial cells was found when 7 was given at a dose of 384 μmol/kg/day×10 days. However, when the dose of 7 was reduced to 192 μmol/kg/d×10 days, there were no drug-related abnormalities.

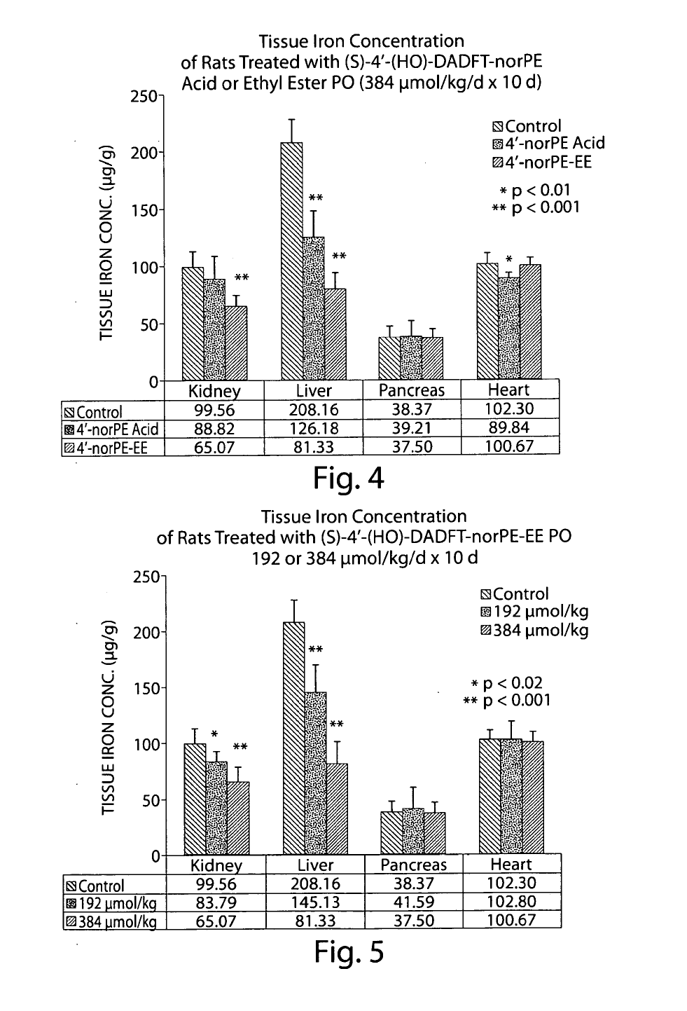

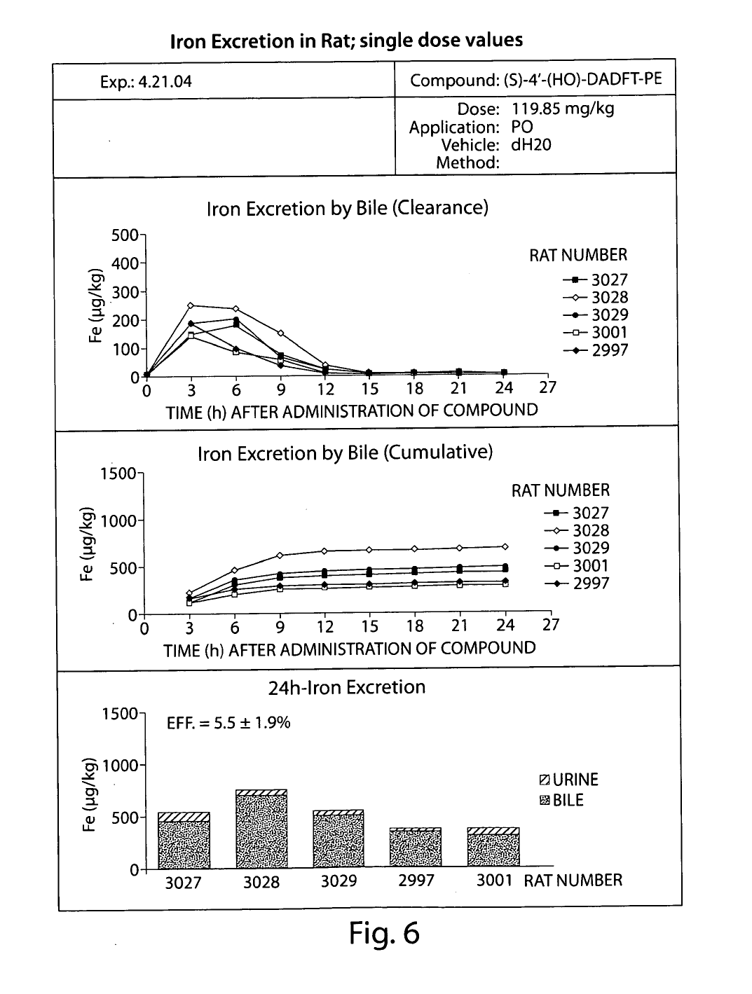

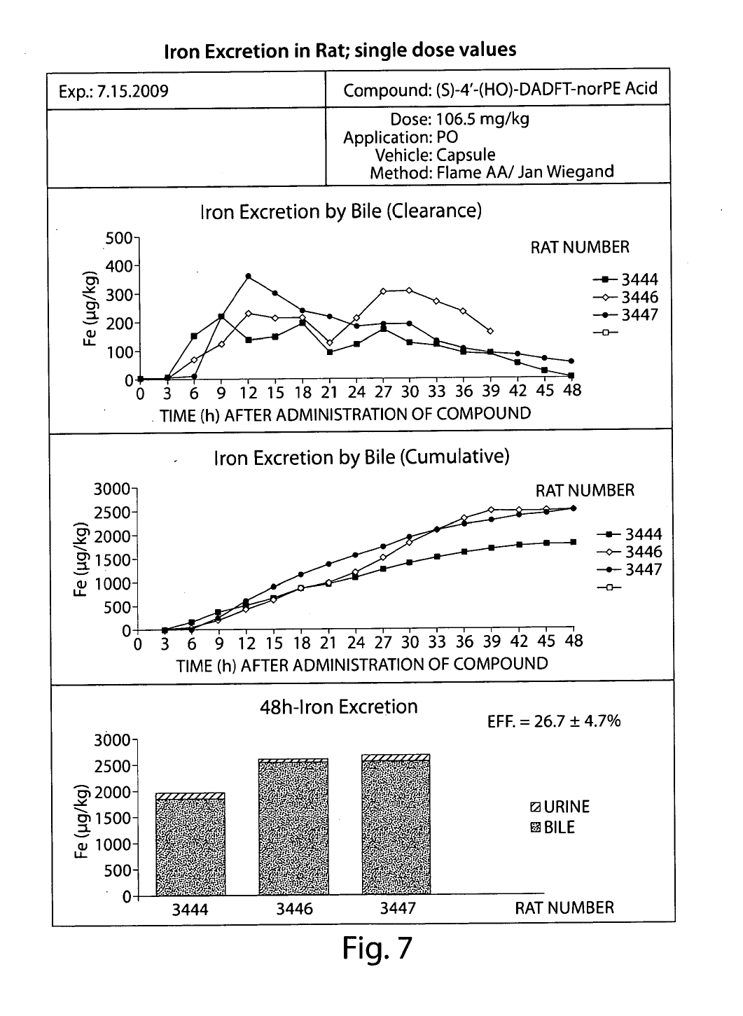

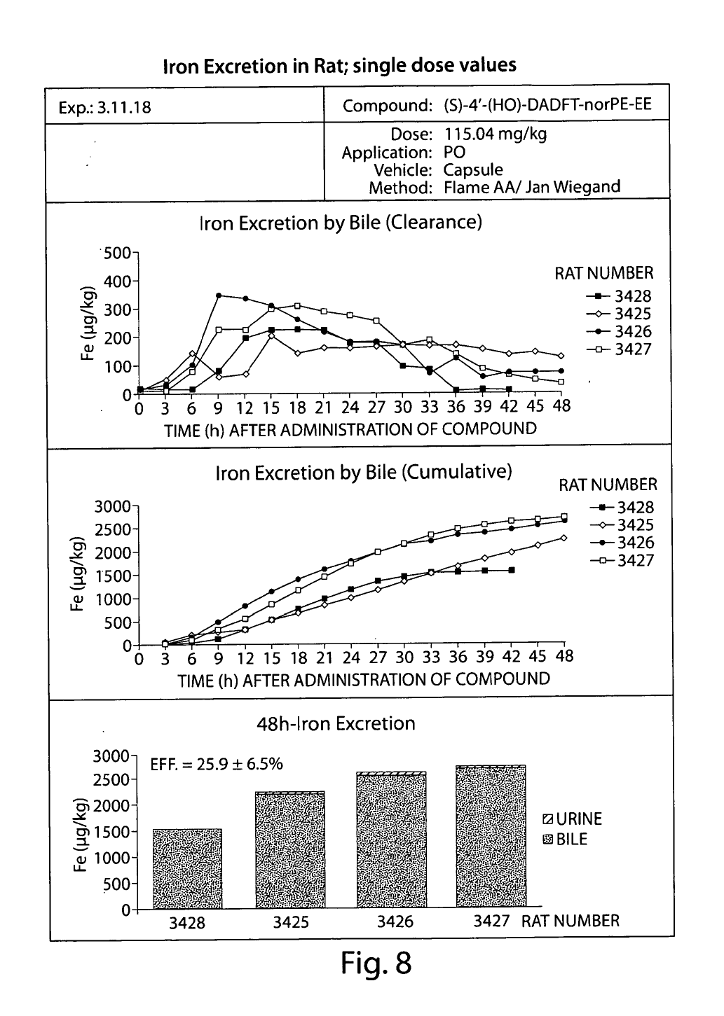

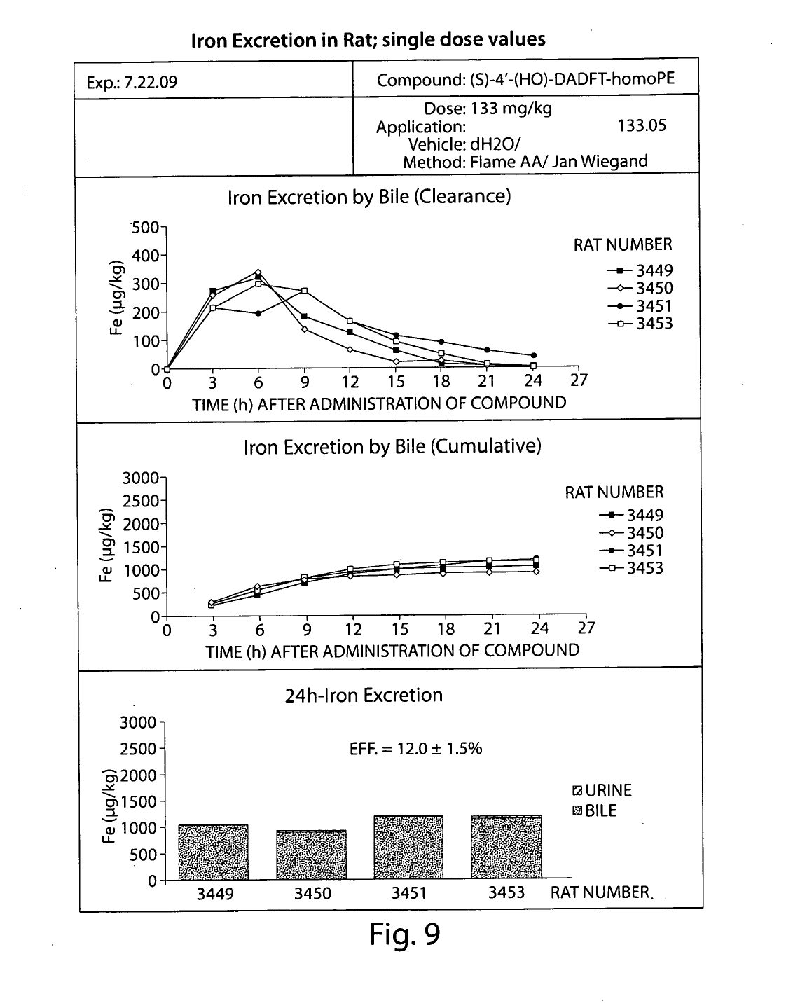

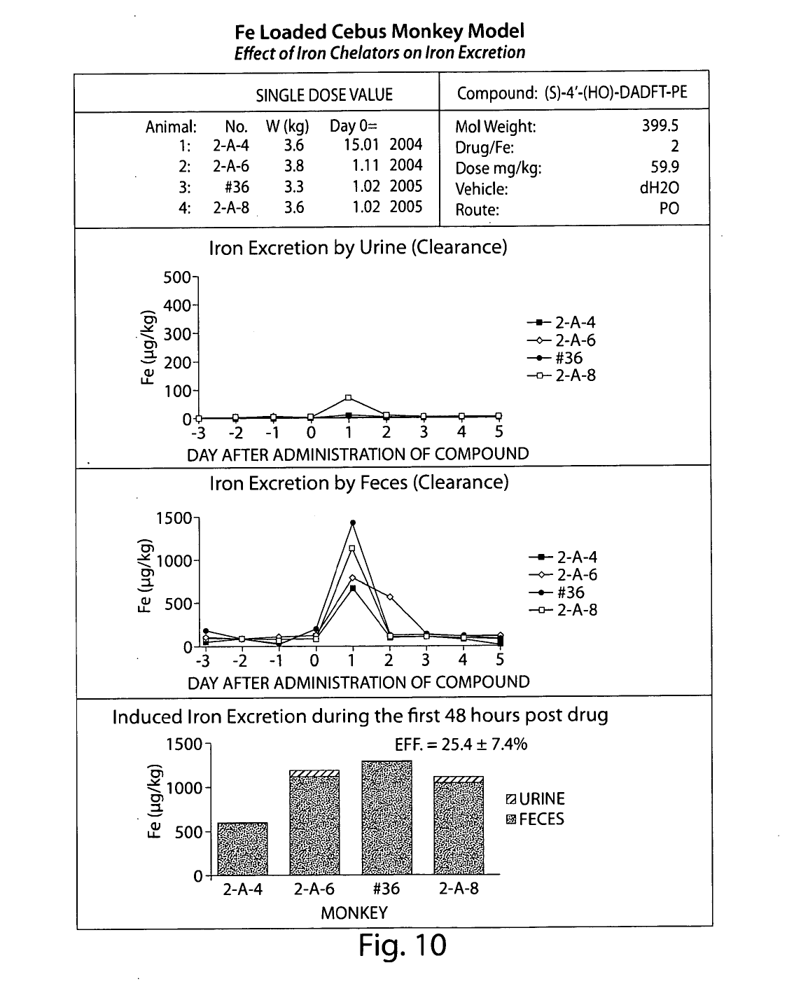

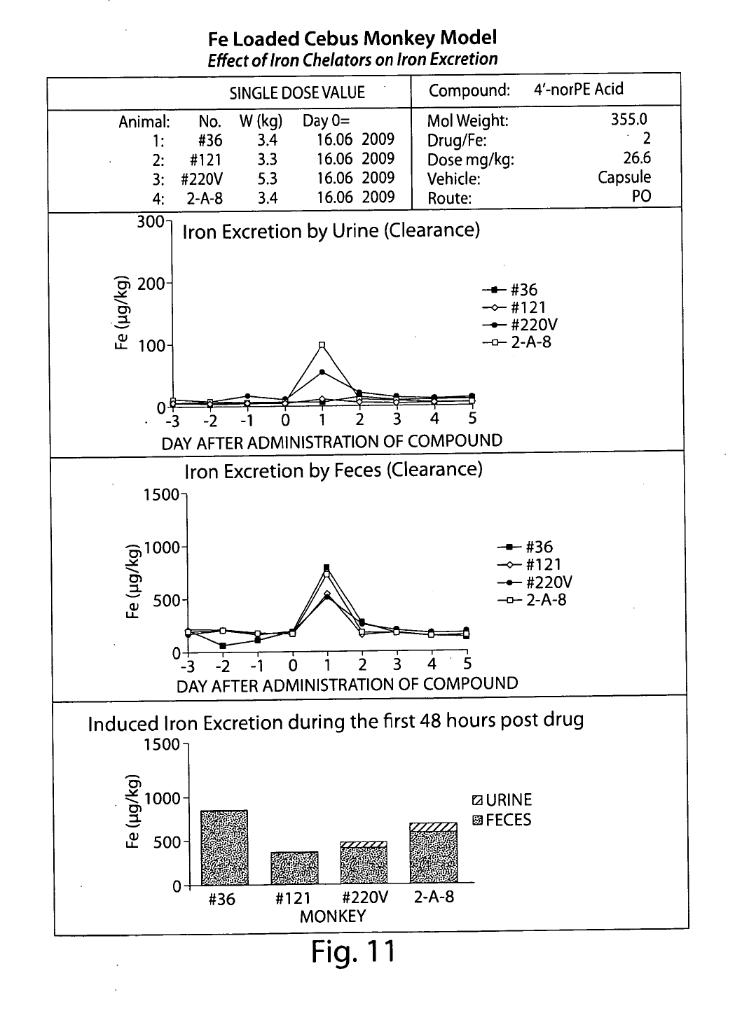

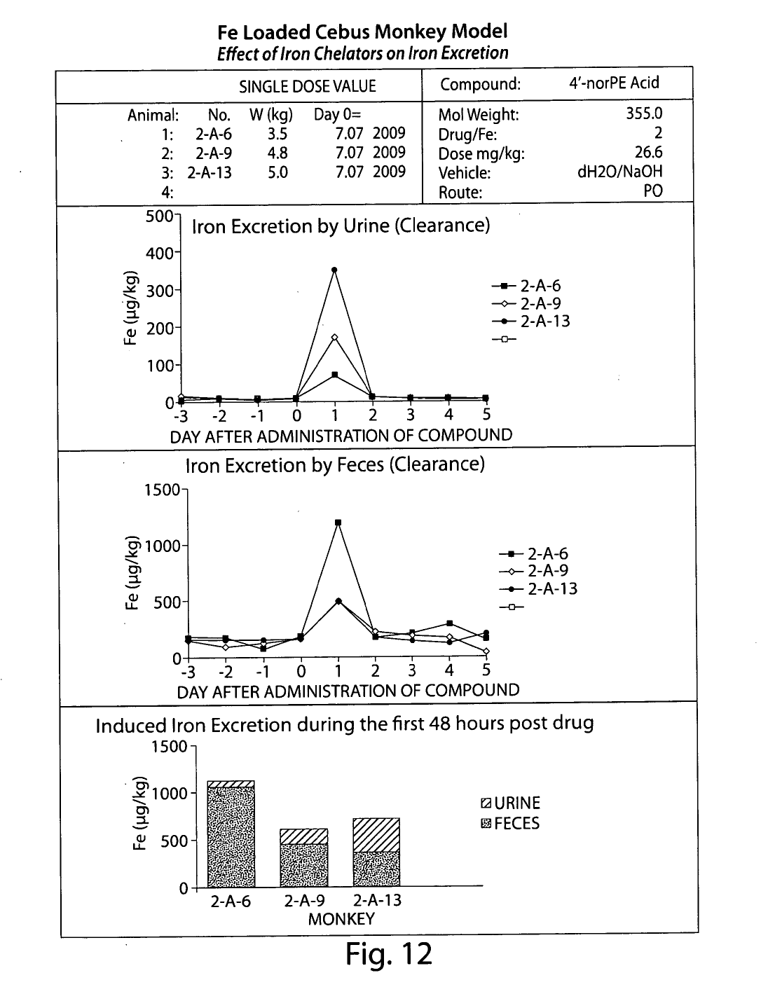

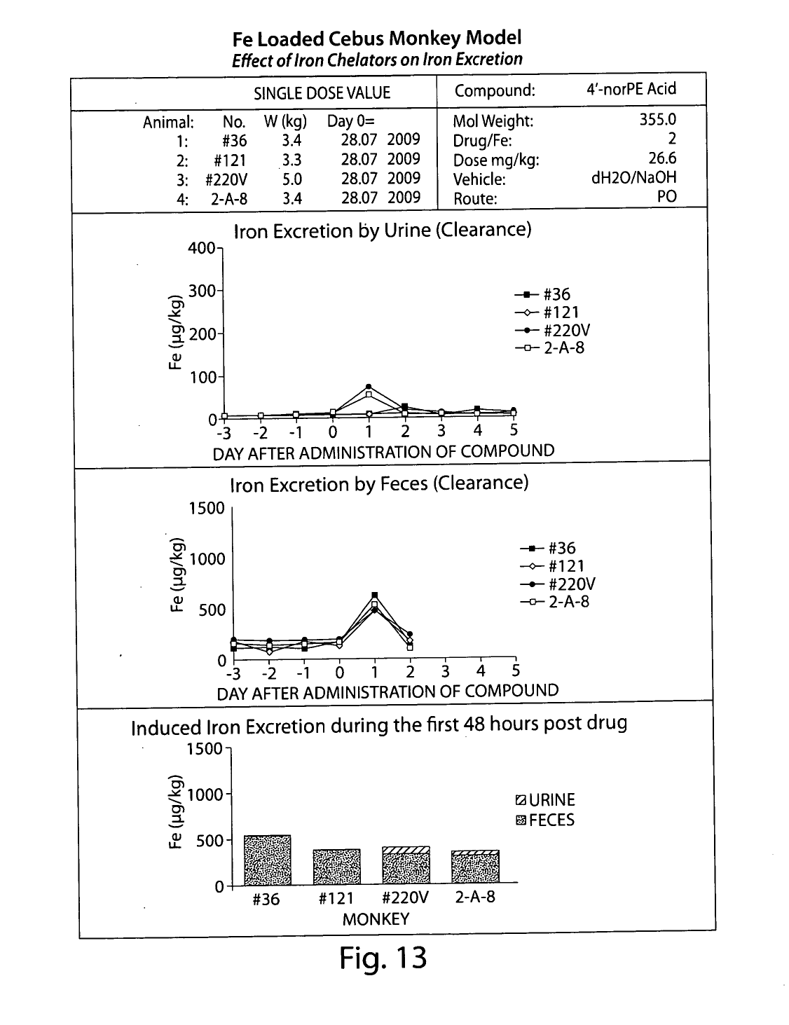

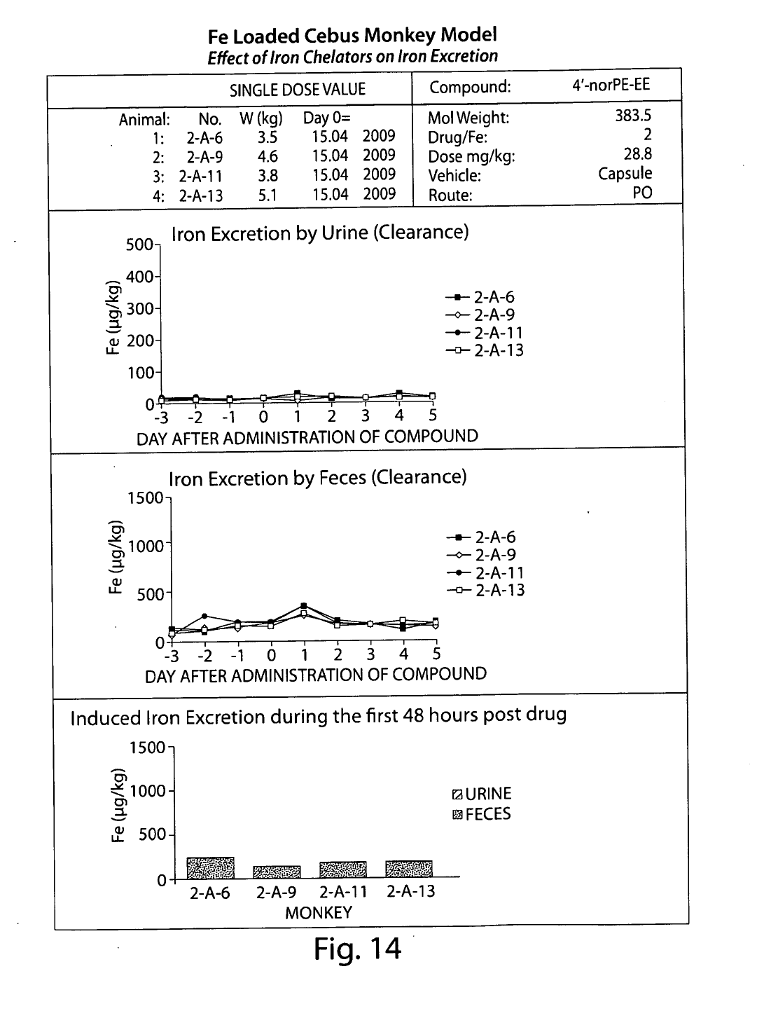

Tissue iron decorporation: As described above, rodents were given acid 6 or 7 orally at a dose of 384 μmol/kg/day×10 days. Ethyl ester 7 was also given at a dose of 192 μmol/kg/d×10 days. On day 11, the animals were euthanized and the kidney, liver, pancreas, and heart were removed. The tissue samples were wet-ashed, and their iron levels were determined (FIGS. 4 and 5). The renal iron content of rodents treated with 6 was reduced by 7.4% when the drug was administered in capsules, and by 24.8% when it given as its sodium salt (FIG. 4). Although the renal iron content of the latter animals was significantly less than that of the untreated controls (p<0.001), there was not a significant difference between the capsule or sodium salt groups (p>0.05). The reduction in liver iron was profound, >35% in both the capsule and sodium salt groups (p<0.001). There was a significant reduction in pancreatic iron when the drug was given as its sodium salt (p<0.05) vs the untreated controls, but not when it was dosed in capsules (FIG. 4). However, as with the renal iron, there was no significant difference between the capsule vs sodium salt treatment groups (p>0.05). Finally, there was a significant decrease in the cardiac iron of animals treated with acid 6, 6.9% and 9.9% when the drug was given in capsules and as its sodium salt, respectively (p<0.05).

Rats given the ethyl ester 7 in capsules orally at a dose of 384 μmmol/kg/day×10 days had a profound reduction in both renal and hepatic iron versus the untreated controls, 32.1% (p<0.001) and 59.1% (p<0.001), respectively (FIG. 5). We have never observed such a dramatic decrease in tissue iron concentration. Due to the renal toxicity observed with 7 at the 384 μmol/kg/d dosing regimen, we decided to repeat the 10-day toxicity study, this time administering the drug at half of the dose, 192 μmmol/kg/d. A clear dose response was observed in the reduction in renal and liver iron concentrations (FIG. 5). The kidney iron reduction was 32.1% at 384 μmol/kg/d, and 12.6% at 192 μmol/kg/d (p<0.01). The liver iron reduction was 59.1% at 384 μmol/kg/d, and 27% at 192 μmol/kg/d (p<0.001). Neither dose was associated with a reduction in pancreatic or cardiac iron content.

Earlier studies with 2 revealed that methylation of the 4′-hydroxyl resulted in a ligand (3) with better ICE in both the rodents and the primates (FIG. 1).⁴³ However, ligand 3 was unacceptably nephrotoxic,³⁴ and was reengineered, adding a 3,6,9-trioxadecyl group to the 4′-(HO) in place of the methyl.^{34,46,47} This resulted in a chelator (4) with about the same ICE in rodents and primates as methylated analogue 3, but virtually absent of any nephrotoxicity.³⁴ The corresponding 3′- and the 5′-trioxa analogues also had better ICE properties in rodents than the 4′-O-methyl ether 3. In the primates, the ICE of the 3′-trioxa ligand was similar to that of the 4′-trioxa analogue (4), while the 5′-was less effective. These data encouraged an assessment of how altering the length of the polyether chain would affect a ligand's ICE, lipophilicity, and physiochemical properties.

The 3,6,9-trioxadecyloxy substituent at the 4′-position of ligand 4³⁴ was both lengthened to a 3,6,9,12-tetraoxamidecyloxy group, providing 5, and shortened to a 3,6-dioxaheptyloxy moiety, providing 6. In addition, the ethyl (7) and isopropyl (13) esters of ligand 6 were also generated. The synthetic methodologies were very simple with high yields, an advantage when large quantities of drug are required for preclinical studies.

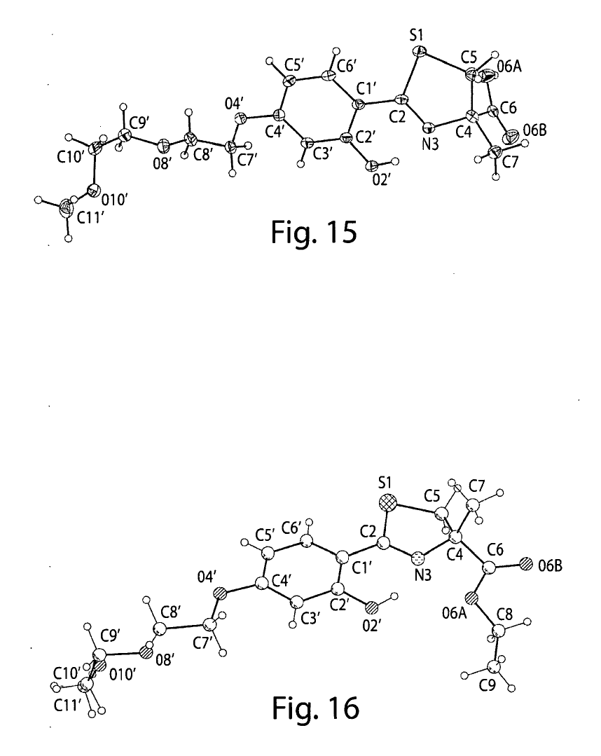

In all cases, the ethyl ester of 2, compound 10, served as the starting material (Schemes 1 and 2). The 4′-(HO) of 10 was alkylated with either polyether iodide 9 or tosylate 12 to afford 11 or 7, respectively. This was followed by hydrolysis of the ethyl ester in aqueous base providing 5 (an oil) with a longer polyether chain (Scheme 3), or ligand 6, possessing a shorter polyether chain (Scheme 4). Both 6 and its ester 7 are crystalline solids. The toxicity profile, efficacy as an iron-clearing agent, and physiochemical state, a crystalline solid, make ligand 6 an attractive clinical candidate. The fact that the ethyl ester of 6, masked ligand 7, also readily crystallizes is remarkable (see X-ray structures, FIGS. 15 and 16). All polyether analogues previously synthesized by this laboratory, both acids and esters, were oils.^{34,46,47} In most instances, metal salts of the former were hygroscopic. Interestingly, even the isopropyl ester of 6, compound 13, was an oil. Since 6 and 7 are crystalline solids, they were given in capsules⁵⁶ to both the rodents and the primates.

In rodents, the ICE of 5 as its sodium salt was nearly 11 times greater than that of the parent (2), and twice as effective as the trioxa polyether (4). The shorter polyether acid 6 given in capsules had an ICE that was 24 times greater than 2, and was nearly five times greater than that of 4 (FIG. 1). The ICE of the corresponding ester 7 was virtually identical to that of 6. The biliary ferrokinetics curves for both 6 and 7 were profoundly different than any of the other ligands (FIG. 2). MIC did not occur until 12-15 h post-drug, and iron clearance was still ongoing even at 48 h. In contrast, MIC occurred much earlier with the other ligands, 3 h for 2 and 4, and 6 h for 5. In addition, iron excretion had returned to baseline levels by 9 h for 2, 12 h for 4 and 24 h for 5 (FIG. 2). If the protracted iron clearance properties of ligand 6 were also observed in humans, thalassemia patients may only need to be treated two to three times a week. This would be an improvement over the rigors of the currently available treatment regimens.

In primates, the ICE of the parent polyether 4 was 2.5 greater than that of the longer analogue 5, while the ICE of the shorter polyether analogue 6 was within error of that of 4 (FIG. 1). However, the ICE of the ethyl ester of 6, ligand 7, is only one third that of 6 (FIG. 1). Studies in rat and monkey plasma suggested no difference in the nonspecific esterase hydrolysis of 7 between the rats and the primates. The poor ICE of 7 in the monkeys is, however, consistent with the idea that the ester is absorbed much more effectively from the GI tract in rodents than in primates.

The protracted biliary ferrokinetics and outstanding iron clearing efficiencies of polyether acid 6 and ester 7 noted in the bile duct-cannulated rats (FIG. 2) were reflected in a dramatic reduction in the tissue iron levels of rodents treated orally with the drugs once daily for 10 days (FIGS. 4 and 5). Acid 6, given orally in capsules, or by gavage as its sodium salt, significantly reduced both hepatic and cardiac iron (FIG. 4) with no histological abnormalities noted between the treated and the control groups. Compound 7 administered in capsules decorporated even more iron from the kidney and liver than 6, but had no impact on pancreatic or cardiac iron burden (FIG. 5). However, ester 7 presented with unacceptable renal toxicity.

Compound 11 (Scheme 3), the ethyl ester of chelator 5 (FIG. 1), was an intermediate in the synthesis of 5. The ester, even if cleaved to the acid 5 in animals by nonspecific serum esterases, would not be expected to perform any better than the parent acid itself. This is underscored when comparing acid 6 (FIG. 1) with its ester 7 (FIG. 1). This ester does not work as well in primates as the parent acid. The synthesis of 13 was simply to assess whether esters other than the ethyl ester of 7 could also be expected to be solids.

Materials. Reagents were purchased from Aldrich Chemical Co. (Milwaukee, Wis.). Fisher Optima grade solvents were routinely used, and DMF was distilled. Reactions were run under a nitrogen atmosphere, and organic extracts were dried with sodium sulfate. Silica gel 40-63 from SiliCycle, Inc. (Quebec City, Quebec, Canada) was used for column chromatography. Melting points are uncorrected. Glassware that was presoaked in 3 N HCl for 15 min, washed with distilled water and distilled EtOH, and oven-dried was used during the isolation of 5 and 6. Optical rotations were run at 589 nm (sodium D line) and 20° C. on a Perkin-Elmer 341 polarimeter, with c being concentration in grams of compound per 100 mL of CHCl₃. ¹H NMR spectra were run in CDCl₃ at 400 MHz, and chemical shifts (δ) are given in parts per million downfield from tetramethylsilane. Coupling constants (J) are in hertz. ¹³C NMR spectra were measured in CDCl₃ at 100 MHz, and chemical shifts (δ) are given in parts per million referenced to the residual solvent resonance of δ 77.16. The base peaks are reported for the ESI-FTICR mass spectra. Elemental analyses were performed by Atlantic Microlabs (Norcross, Ga.) and were within ±0.4% of the calculated values. Purity of the compounds is supported by elemental analyses and high pressure liquid chromatography (HPLC). In every instance, the purity was ≧95%.

Cebus apella monkeys were obtained from World Wide Primates (Miami, Fla.). Male Sprague-Dawley rats were procured from Harlan Sprague-Dawley (Indianapolis, Ind.). Ultrapure salts were obtained from Johnson Matthey Electronics (Royston, UK). All hematological and biochemical studies⁴¹ were performed by Antech Diagnostics (Tampa, Fla.). Atomic absorption (AA) measurements were made on a Perkin-Elmer model 5100 PC (Norwalk, Conn.). Histopathological analysis was carried out by Florida Vet Path (Bushnell, Fla.).

Synthesis of Ethyl (S)-4,5-dihydro-2-[2-hydroxy-4-(3,6-dioxaheptyloxy)phenyl]-4-methyl-4-thiazolecarboxylate (7). Activated K₂CO₃ (2.16 g, 15.64 mmol) and the tosylate (12) (3.97 g, 14.50 mmol) were added to (10) (see WO 2006/107626) (4.0 g, 14.22 mmol) in dry acetone (100 mL). The reaction mixture was heated at reflux for 2 days. After cooling to room temperature the solids were filtered and the solvent was removed under vacuum. The residue was dissolved in 1:1 0.5 M citric acid/saturated NaCl (100 mL) and was extracted with EtOAc (3×50 mL). Combined organic extracts were washed with distilled H₂O (100 mL) and saturated brine (100 mL). The solvent was removed under vacuum providing colorless oil. The oil was crystallized in EtOAc/Hexame to furnish 3.97 g of 4 (73%) as white solid, mp 68-70° C.; ¹H NMR δ 1.30 (t, 3H, J=7.2), 1.66 (s, 3H), 3.19 (d, 1H, J=11.2), 3.40 (s, 3H), 3.57-3.59 (m, 2H), 3.71-3.73 (m, 2H), 3.83-3.88 (d+m, 3H, J=11.6), 4.16 (t, 2H, J=4.8), 4.24 (dq, 2H, J=7.2), 6.46 (dd, 1H, J=2.4, 8.8), 6.49 (d, 1H, J=2.8), 7.29 (d, 1H J=8.4); 100 MHz ¹³C NMR δ 14.12, 24.48, 39.84, 59.09, 61.89, 67.55, 69.52, 70.80, 71.94, 83.12, 101.45, 107.28, 109.89, 131.69, 161.18, 162.99, 170.81, 172.80; HRMS m/z calcd for C₁₈H₂₆NO₆S, 384.1475 (M+H). found, 384.1509.

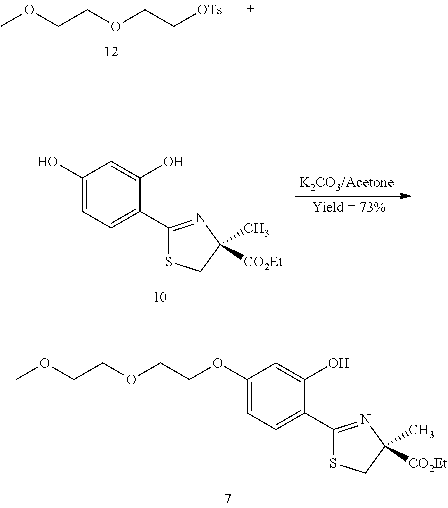

Synthesis of (S)-4,5-Dihydro-2-[2-hydroxy-4-(3,6-dioxaheptyloxy)phenyl]-4-methyl-4-thiazolecarboxylic acid (6). A solution of 50% (w/w) NaOH (2.1 mL, 40 mmol) in CH₃OH (20 mL) was added to (7) (1.2 g, 3.1 mmol) in 30 mL CH₃OH at 0° C. The reaction mixture was stirred at room temperature for 6 h, and the bulk of the solvent was removed under vacuum. The residue was treated with dilute NaCl (30 mL) and extracted with ether (2×20 mL). The aqueous layer was cooled in ice, acidified with 6 N HCl to pH=2, and extracted with EtOAc (4×25 mL). EtOAc layers were washed with saturated NaCl (50 mL). When run extraction of chelator all glassware were first soaked in 3 N HCl for 15 min to remove any extraneous iron. The solvent was removed providing light pale colored oil, which was crystallized in EtOAc/Hexane to furnish 0.880 g of 1 (80%) as solid, mp 82-83° C.; ¹H NMR δ 1.70 (s, 3H), 3.22 (d, 1H J=11.2), 3.40 (S, 3H), 3.58-3.60 (m, 2H), 3.71-3.73 (m, 2H), 3.83-3.87 (m, 3H), 4.15 (t, 2H, J=5.2), 6.45 (dd, 1H, J=2.0, 8.8), 6.51 (d, 1H, J=2.0), 7.28 (d, 1H, J=8.4); 100 Mhz ¹³C NMR δ 24.58, 39.77, 59.13, 67.64, 69.61, 70.77, 71.99, 82.63, 101.53, 107.73, 109.63, 131.88, 161.42, 163.40, 171.96, 176.91; HRMS m/z calcd for C₁₆H₂₂NO₆S, 356.1162 (M+H). found, 356.1190.

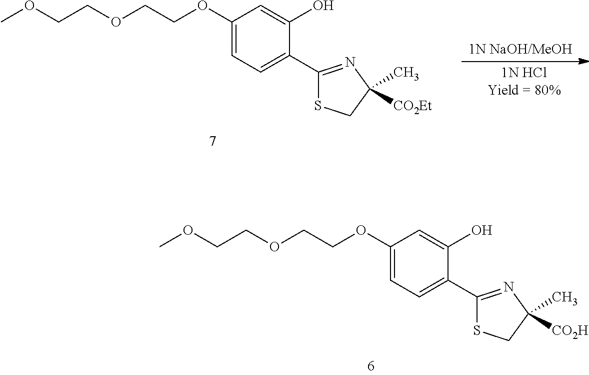

Synthesis of (S)-4,5-Dihydro-2-[2-hydroxy-4-(3,6,9,12-tetraoxamidecyloxy)phenyl]-4-methyl-4-thiazolecarboxylic Acid (5). A solution of 50% (w/w) NaOH (7.0 g, 87 mmol) in CH₃OH (75 mL) was added to 11 (3.64 g, 7.72 mmol) in CH₃OH (85 mL) at 0° C. over 3 min. The reaction mixture was stirred at 0° C. for 1.5 h and at room temperature for 18 h, and the bulk of the solvent was removed under reduced pressure. The residue was treated with H₂O (90 mL) and was extracted with CHCl₃ (4×50 mL). The aqueous layer was cooled in ice, combined with saturated NaCl (45 mL) and cold 5 N HCl (22 mL), and was extracted with EtOAc (100 mL, 5×70 mL). The EtOAc layers were washed with saturated NaCl (75 mL). Solvent was removed in vacuo, affording 3.20 g of 5 (94%) as a yellow oil: [α]+47.6° (c 0.86). ¹H NMR (CDCl₃+1-2 drops D₂O) δ 1.69 (s, 3H), 3.21 (d, 1H, J=11.3), 3.38 (s, 3 H), 3.53-3.57 (m, 2H), 3.62-3.69 (m, 8H), 3.70-3.73 (m, 2H), 3.82-3.87 (m, 3H), 4.11-4.15 (m, 2H), 6.45 (dd, 1H, J=8.8, 2.5), 6.50 (d, 1H, J=2.4), 7.27 (d, 1H, J=9.0). ¹³C NMR δ 24.67, 39.90, 59.11, 69.66, 70.53, 70.67, 70.69, 70.71, 70.94, 72.02, 82.93, 101.56, 107.70, 109.80, 131.85, 161.32, 163.30, 171.76, 176.19. HRMS m/z calcd for C₂₀H₃₀NO₈S, 444.1687 (M+H); found, 444.1691. Anal. (C₂₀H₂₉NO₈S.0.5H₂O) C, H, N.

Synthesis of (S)-4,5-Dihydro-2-[2-hydroxy-4-(3,6-dioxaheptyloxy)phenyl]-4-methyl-4-thiazolecarboxylic Acid (6). A solution of 50% (w/w) NaOH (2.1 mL, 40 mmol) in CH₃OH (20 mL) was added to 7 (1.2 g, 3.1 mmol) in CH₃OH (30 mL) at 0° C. The reaction mixture was stirred at room temperature for 6 h, and the bulk of the solvent was removed under reduced pressure. The residue was treated with dilute NaCl (30 mL) and was extracted with ether (2×20 mL). The aqueous layer was cooled in ice, acidified with 6 N HCl to pH=2, and extracted with EtOAc (4×25 mL). The EtOAc layers were washed with saturated NaCl (50 mL). Solvent was removed in vacuo, and recrystallization from EtOAc/hexanes furnished 0.880 g of 6 (80%) as a solid, mp 82-83° C.: [α]+59.6° (c 0.094). ¹H NMR δ 1.70 (s, 3H), 3.22 (d, 1H, J=11.2), 3.40 (s, 3H), 3.58-3.60 (m, 2H), 3.71-3.73 (m, 2H), 3.83-3.87 (m+d, 3H, J=12.0), 4.15 (t, 2H, J=5.2), 6.45 (dd, 1H, J=8.8, 2.0), 6.51 (d, 1H, J=2.0), 7.28 (d, 1H, J=8.4). ¹³C NMR δ 24.58, 39.77, 59.13, 67.64, 69.61, 70.77, 71.99, 82.63, 101.53, 107.73, 109.63, 131.88, 161.42, 163.40, 171.96, 176.91. HRMS m/z calcd for C₁₆H₂₂NO₆S, 356.1162 (M+H); found, 356.1190. Anal. (C₁₆H₂₁NO₆S) C, H, N.

Synthesis of Ethyl (S)-4,5-dihydro-2-[2-hydroxy-4-(3,6-dioxaheptyloxy)phenyl]-4-methyl-4-thiazolecarboxylate (7). Flame activated K₂CO₃ (2.16 g, 15.6 mmol) and 12⁴⁹ (3.97 g, 14.5 mmol) were added to 10⁴⁸ (4.0 g, 14.2 mmol) in acetone (100 mL). The reaction mixture was heated at reflux for 2 d. After cooling to room temperature, the solids were filtered and washed with acetone, and the filtrate was concentrated by rotary evaporation. The residue was treated with 1:1 0.5 M citric acid/saturated NaCl (100 mL) and was extracted with EtOAc (3×50 mL). The organic extracts were washed with H₂O (100 mL) and saturated NaCl (100 mL). After solvent was removal in vacuo, recrystallization from EtOAc/hexanes furnished 3.97 g of 7 (73%) as a solid, mp 68-70° C.: [α]+47.4° (c 0.114). ¹H NMR δ 1.30 (t, 3H, J=7.2), 1.66 (s, 3H), 3.19 (d, 1H, J=11.2), 3.40 (s, 3H), 3.57-3.59 (m, 2H), 3.71-3.73 (m, 2H), 3.83-3.88 (d+m, 3H, J=11.6), 4.16 (t, 2H, J=4.8), 4.24 (dq, 2H, J=7.2, 1.6), 6.46 (dd, 1H, J=8.8, 2.4), 6.49 (d, 1H, J=2.8), 7.29 (d, 1H, J=8.4), 12.69 (s, 1H). ¹³C NMR δ 14.12, 24.48, 39.84, 59.09, 61.89, 67.55, 69.52, 70.80, 71.94, 83.12, 101.45, 107.28, 109.89, 131.69, 161.18, 162.99, 170.81, 172.80. HRMS m/z calcd for C₁₈H₂₆NO₆S, 384.1475 (M+H); found, 384.1509. Anal. (C₁₈H₂₅NO₆S) C, H, N.

Synthesis of 13-Iodo-2,5,8,11-tetraoxamidecane (9). Sodium iodide (8.61 g, 57.5 mmol) was added to a solution of 8 (10.37 g, 28.61 mmol) in acetone (230 mL), and the reaction mixture was heated at reflux for 18 h. After the solvent was evaporated in vacuo, the residue was combined with H₂O (150 mL) and was extracted with CH₂Cl₂ (150 mL, 2×80 mL). The organic extracts were washed with 1% NaHSO₃ (80 mL), H₂O (80 mL), and saturated NaCl (50 mL), and solvent was evaporated in vacuo. Purification by flash column chromatography using 14% acetone/CH₂Cl₂ generated 8.56 g of 9 (94%) as a colorless liquid: ¹H NMR δ 3.24-3.29 (m, 2H), 3.39 (s, 3H), 3.54-3.58 (m, 2H), 3.64-3.70 (m, 10H), 3.74-3.78 (m, 2H). ¹³C NMR S 59.17, 70.32, 70.65, 70.70, 70.73, 70.77, 72.05, 72.09. HRMS m/z calcd for C₉H₂₀IO₄, 319.0401 (M+H); found, 319.0417. Anal. (C₉H₁₉IO₄) C, H.

Synthesis of Ethyl (S)-4,5-dihydro-2-[2-hydroxy-4-(3,6,9,12-tetraoxamidecyloxy)phenyl]-4-methyl-4-thiazolecarboxylate (11). Flame activated K₂CO₃ (0.666 g, 4.82 mmol) was added to a solution of 9 (1.46 g, 4.59 mmol) and 10⁴⁸ (1.08 g, 3.84 mmol) in acetone (85 mL), and the reaction mixture was heated at reflux for 43 h. After cooling to room temperature, the solids were filtered and washed with acetone, and the filtrate was concentrated by rotary evaporation. The residue was combined with 1:1 0.5 M citric acid/saturated NaCl (100 mL) and was extracted with EtOAc (3×80 mL). The organic extracts were washed with 1% NaHSO₃ (80 mL), H₂O (80 mL), and saturated NaCl (55 mL). After solvent was removal in vacuo, the residue was purified by flash column chromatography using 25% acetone/petroleum ether then 9% acetone/CH₂Cl₂, furnishing 1.33 g of 11 (73%) as a yellow oil: [α]+36.2° (c 1.20). ¹H NMR δ 1.30 (t, 3H, J=7.2), 1.66 (s, 3H), 3.19 (d, 1H, J=11.3), 3.38 (s, 3H), 3.52-3.56 (m, 2H), 3.62-3.74 (m, 10H), 3.81-3.88 (m, 3H), 4.12-4.16 (m, 2H), 4.20-4.28 (m, 2H), 6.46 (dd, 1H, J=8.6, 2.3), 6.49 (d, 1 H, J=2.4), 7.29 (d, 1H, J=8.6). ¹³C NMR δ 14.21, 24.59, 39.95, 59.14, 62.01, 67.66, 69.58, 70.62, 70.71, 70.73, 70.97, 72.04, 83.23, 101.52, 107.42, 109.99, 131.78, 161.28, 163.109, 170.90, 172.95. HRMS m/z calcd for C₂₂H₃₄NO₈S, 472.2000 (M+H); found, 472.2007. Anal. (C₂₂H₃₃NO₈S) C, H, N.

Synthesis of Isopropyl (S)-4,5-dihydro-2-[2-hydroxy-4-(3,6-dioxaheptyloxy)phenyl]-4-methyl-4-thiazolecarboxylate (13). 2-Iodopropane (1.60 g, 9.41 mmol) and DIEA (1.22 g, 9.44 mmol) were successively added to 6 (2.1 g, 5.9 mmol) in DMF (50 mL), and the reaction mixture was stirred at room temperature for 72 h. After solvent removal under high vacuum, the residue was treated with 1:1 0.5 M citric acid/saturated NaCl (100 mL) and was extracted with EtOAc (3×100 mL). The organic extracts were washed with 50 mL portions of 1% NaHSO₃, H₂O, and saturated NaCl, and solvent was evaporated in vacuo. Purification by flash column chromatography using 5% acetone/CH₂Cl₂ generated 1.99 g of 13 (85%) as a yellow oil: [α]+40.0° (c 0.125). ¹H NMR δ 1.26 and 1.27 (2 d, 6H, J=5.5), 1.63 (s, 3H), 3.17 (d, 1H, J=11.2), 3.38 (s, 3H), 3.55-3.58 (m, 2H), 3.69-3.72 (m, 2H), 3.81-3.86 (d+m, 3H, J=11.2), 4.15 (t, 2H, J=5.2), 5.07 (septet, 1H, J=6.4), 6.46 (dd, 1H, J=9.2, 2.0), 6.49 (d, 1H, J=2.4), 7.28 (d, 1H, J=8.4), 12.7 (br s, 1H). ¹³C NMR δ 21.54, 24.27, 39.63, 58.98, 67.46, 69.35, 69.42, 70.69, 71.85, 83.10, 101.37, 107.14, 109.83, 131.57, 161.11, 162.88, 170.55, 172.10. HRMS m/z calcd for C₁₉H₂₈NO₆S, 398.1637 (M+H); found, 398.1658. Anal. (C₁₉H₂₇NO₆S) C, H, N.

X-ray experimental data for compounds (6) and (7). X-ray data were collected at 173 K on a Siemens SMART PLATFORM equipped with A CCD area detector and a graphite monochromator utilizing MoK_{α} radiation (λ=0.71073 Å). Cell parameters were refined using up to 8192 reflections. A full sphere of data (1850 frames) was collected using the ω-scan method (0.3° frame width). The first 50 frames were re-measured at the end of data collection to monitor instrument and crystal capability (maximum correction on I was <1%). Absorption corrections by integration were applied based on measured indexed crystal faces.

The structures were solved by the Direct Methods in SHELXTL6,⁵⁷ and refined using full-matrix least squares. The non-H atoms were treated anisotropically, whereas the hydrogen atoms were calculated in ideal positions and were riding on their respective carbon atoms. For 6, a total of 227 parameters were refined in the final cycle of refinement using 3588 reflections with I>2σ(I) to yield R₁ and wR₂ of 3.16% and 8.58%, respectively. For compound 7, a total of 243 parameters were refined in the final cycle of refinement using 4082 reflections with I>2σ(I) to yield R₁ and wR₂ of 2.52% and 6.53%, respectively. Refinements were done using F². Full crystallographic data for 6 and 7 have been submitted to CCDC (deposition nos. CCDC 757291 & 757292).

Iron clearing efficiency of iron chelators in a non-iron overloaded, bile duct cannulated rat model. Studies are performed in the non-iron overloaded, bile duct cannulated rodent model with the compounds of the invention. Briefly, male Sprague-Dawley rats averaging 450 g are housed in Nalgene plastic metabolic cages during the experimental period and given free access to water. The animals are anesthetized using sodium pentobarbital (55 mg/kg) administered intraperitoneally. The bile duct is cannulated using 22-gauge polyethylene tubing. The cannula is inserted into the duct about 1 cm from the duodenum and tied snugly in place. After threading through the shoulder, the cannula ifs passed from the rat to the swivel inside a metal torque-transmitting tether, which is attached to a rodent jacket around the animal's chest. The cannula is directed from the rat to a Gilson microfraction collector (Middleton, Wis.) by a fluid swivel mounted above the metabolic cage. Three hour bile samples are continuously collected for a minimum of 24 hours up to 48 hours. However, the efficiency calculations are based on the 24 hour iron excretion. The efficiency of each chelator is calculated on the basis of a 2:1 ligand-iron complex. The efficiencies in the rodent model are calculated by subtracting the iron excretion of control animals from the iron excretion of treated animals. This number is then divided by the theoretical output; the result is expressed as a percentage (Bergeron et al. J. Med. Chem. 1999, 42, 95-108) the entire contents of which are incorporated herein by reference). Data are presented as the mean±the standard error of the mean; p-values were generated via a one-tailed Student's t-test in which the inequality of variances was assumed; and a p-value of <0.05 was considered significant. The urine sample is taken at 24 hours and handled as previously described in Bergeron et al. J. Med. Chem. 1991, 34, 2072-2078, the entire contents of which are incorporated herein by reference.

Iron chelators in a Cebus apella monkey model. Studies are performed in the iron-overloaded monkey model with the compounds of the invention. The protocol used can be found in Bergeron et al. J. Med. Chem. 2003, 46, 1470-1477, the contents of which are incorporated herein by reference. Briefly, the monkeys are iron overloaded with iron dextran administered intravenously to result in an iron loading of about 500 mg per kg of body weight. At least 20 half-lives, 60 days, elapse before the animals are used in experiments evaluating iron chelators. The iron chelators are suspended in vehicle and administered either p.o. or s.c. Fecal and urine samples are collected at 24 hour intervals beginning 4 days prior to the administration of an iron chelator and continued for 5 days after the chelator is administered. Iron concentrations in stool and urine are determined by flame atomic absorption spectroscometry. Iron chelator efficiency is calculated by dividing the net iron clearance [total iron excretion (stool plus urine) minus background] by the theoretical iron clearance and multiplying by 100. The theoretical clearance of the iron chelator is generated on the basis of a 2:1 ligand/iron complex.

Tissue distribution upon subcutaneous administration to rats. A measurement is made assessing compounds of the invention tissue and plasma concentrations upon subcutaneous administration at times from 2-8 h post dosing. The rats are given the compound subcutaneously at 300 μmol/kg. The tissue and plasma level are obtained as described in Bergeron et al. J. Med. Chem. 2005, 48, 821-831, the entire contents of which are incorporated herein by reference.

Uranium excretion in rats by iron chelators. Male Sprague-Dawley rats averaging 450 g are anesthetized using sodium pentobarbital (55 mg/kg) administered intraperitoneally. The bile duct is cannulated using 22-gauge polyethylene tubing. The rats are given uranyl acetate subcutaneously at 5 mg/kg. Immediately thereafter, the rats are given the chelator intraperitoneally at a dose of 300 μmmol/kg. 24-h urine and 24-h bile samples are collected, acidified with 2% concentrated nitric acid and assessed by Inductively Coupled Plasma Mass Spectrometry (ICP-MS) for their uranium content.

Drug preparation and administration. In the iron clearing experiments, the rats were given 5-7 orally at a dose of 300 μmol/kg. Ligand 5 was given by gavage as its monosodium salt (prepared by the addition of 1 equiv of NaOH to a suspension of the free acid in distilled water), while 6 and 7 were given in capsules. The primates were given 5-7 orally at a dose of Ligand 5 was given to the primates by gavage as its monosodium salt. Analogue 6 was given to the monkeys by gavage as its monosodium salt, as well as in capsules. Ligand 7 was given to the monkeys in capsules. Drug preparation for the rodent toxicity studies of 6 and 7 are described below.

Plasma analytical methods. Analytical separation was performed on a Discovery RP Amide C16 HPLC system with a Shimadzu SPD-10A UV-VIS detector at 310 nm as previously described.^{51,58} Mobile phase and chromatographic conditions were as follows: Mobile Phase A (MPA): 25 mM KH₂PO₄+2.5 mM 1-octanesulfonic acid, pH 3 (95%) and acetonitrile (5%); Mobile Phase B (MPB): 25 mM KH₂PO₄+2.5 mM 1-octanesulfonic acid, pH 3 (40%) and acetonitrile (60%). The chelator concentrations were calculated from the peak area fitted to calibration curves by non-weighted least-squares linear regression with Shimadzu CLASS-NP 7.4 Chromatography Software. The method had a detection limit of 0.1 μM and was reproducible and linear over a range of 0.2-20 μM.

The ethyl ester (7) was solubilized in DMSO and further diluted with distilled water to provide a 100 μM solution. A 25 μL aliquot of the drug solution was added to centrifuge tubes containing 100 μL of rat or primate plasma. Control experiments were also performed in which saline was used in place of the rat or monkey plasma. The centrifuge tubes were vortexed and incubated in a shaking incubator at 37° C. for 1 or 2 h. Note that separate samples were processed for each species at each time point (4 samples total). Methanol (400 μL) was added to the centrifuge tubes at the end of the incubation period to stop the reaction. The tubes were stored at −20° C. for at least 0.5 h. The tubes were then allowed to warm to room temperature. The samples were vortexed and centrifuged for 10 min at 10,000 rpm. Supernatant (100 μL) was diluted with MPA (minus the 1-octanesulfonic acid, 400 μL), vortexed, and run on the HPLC as usual.

Toxicity evaluation of compounds (6) and (7) in rodents. Male Sprague-Dawley rats (300-350 g) were fasted overnight and were given the chelators first thing in the morning. The rats were fed ˜3 h post-drug and had access to food for ˜5 h before being fasted overnight. Ligand 6 was given to the rats orally once daily at a dose of 384 μmol/kg/d×10 d. Note that this dose is equivalent to 100 mg/kg/day of the DFT sodium salt. The chelator (6) was administered orally in gelatin capsules (n=5), or by gavage as its monosodium salt (n=10). The ethyl ester (7) was administered orally in capsules once daily at a dose of 192 (n=6) or 384 μmol/kg/d (n=5)×10 d. Age-matched rats (n=12) served as untreated controls. The rats were euthanized 24 h post-drug (day 11) and extensive tissues were collected for histopathological analysis. Samples of the kidney, liver, heart and pancreas were reserved and assessed for their iron content.

Preparation of rodent tissues for the determination of their iron content. The initial step in the tissue preparation involved removing any obvious membranes or fat. A sample of each tissue (300-350 mg) was weighed and transferred to acid-washed hydrolysis (pressure) tubes. Note that the same region of each tissue was always utilized. Concentrated HNO₃ (65%), 1.5 mL, and distilled water (2 mL) were added. The tubes were then sealed and placed in a 120° C. oil bath for 5 h; the tubes were vented as necessary. Then, the tubes were removed from the oil bath and allowed to cool to room temperature. The temperature of the oil bath was decreased to 100° C. Once the samples were cooled, 0.7 mL of hydrogen peroxide (30%) was added to the hydrolysis tube. The samples were placed back in the oil bath and cooked overnight. The samples were then removed from the oil bath and allowed to cool to room temperature. The hydrolysis tubes were vortexed and the digested samples were poured into 50-mL volumetric flasks. The samples were brought to volume using distilled water. Finally, the samples were poured into a syringe and filtered using 0.45μ 30 mm, Teflon syringe filters. Iron concentrations were determined by flame absorption spectroscopy as presented in other publications.^{40,41}

It will be clear that the invention may be practiced other than as particularly described in the foregoing description and examples. Numerous modifications and variations of the present disclosure are possible in light of the above teachings and, therefore, are within the scope of the claims. Preferred features of each aspect of the disclosure are as for each of the other aspects mutatis mutandis. The documents including patents, patent applications, journal articles, or other disclosures mentioned herein are hereby incorporated by reference in their entirety. In the event of conflict, the disclosure of the present application controls, other than in the event of clear error.

### REFERENCES

1. Graf, E; Mahoney, J. R.; Bryant, R. G.; Eaton, J. W. Iron-Catalyzed Hydroxyl Radical Formation. Stringent Requirement for Free Iron Coordination Site. J. Biol. Chem. 1984, 259, 3620-3624.
2. Halliwell, B. Free Radicals and Antioxidants; A Personal View. Nutr. Rev. 1994, 52, 253-265.
3. Halliwell, B. Iron, Oxidative Damage, and Chelating Agents. In The Development of Iron Chelators for Clinical Use; Bergeron, R. J., Brittenham, G. M., Eds.; CRC: Boca Raton, 1994; pp. 33-56.
4. Koppenol, W. Kinetics and Mechanism of the Fenton Reaction: Implications for Iron Toxicity. In Iron Chelators: New Development Strategies; Badman, D. G.; Bergeron, R. J.; Brittenham, G. M.; Eds.; Saratoga: Ponte Vedra Beach, Fla., 2000, pp 3-10.
5. Babbs, C. F. Oxygen Radicals in Ulcerative Colitis. Free Radical Biol. Med. 1992, 13, 169-181.
6. Hazen, S. L.; d'Avignon, A.; Anderson, M. M.; Hsu, F. F.; Heinecke, J. W. Human Neutrophils Employ the Myeloperoxidase-Hydrogen Peroxide-Chloride System to Oxidize α-Amino Acids to a Family of Reactive Aldehydes. Mechanistic Studies Identifying Labile Intermediates along the Reaction Pathway. J. Biol. Chem. 1998, 273, 4997-5005.
7. Millán, M.; Sobrino, T.; Arenillas, J. F.; Rodríguez-Yáñez, M.; García, M.; Nombela, F.; Castellanos, M.; de la Ossa, N. P.; Cuadras, P.; Serena, J.; Castillo, J.; Dávalos, A. Biological Signatures of Brain Damage Associated with High Serum Ferritin Levels in Patients with Acute Ischemic Stroke and Thrombolytic Treatment. Dis. Markers. 2008, 25, 181-188.
8. Zecca, L.; Casella, L.; Albertini, A.; Bellei, C.; Zucca, F. A.; Engelen, M.; Zadlo, A.; Szewczyk, G.; Zareba, M.; Sarna, T. Neuromelanin Can Protect Against Iron-Mediated Oxidative Damage in System Modeling Iron Overload of Brain Aging and Parkinson's Disease. J. Neurochem. 2008, 106, 1866-1875.
9. Pietrangelo, A. Iron Chelation beyond Transfusion Iron Overload. Am. J. Hematol. 2007, 82, 1142-1146.
10. Pippard, M. J. Iron Overload and Iron Chelation Therapy in Thalassaemia and Sickle Cell Haemoglobinopathies. Acta. Haematol. 1987, 78, 206-211.
11. Olivieri, N. F. Progression of Iron Overload in Sickle Cell Disease. Semin. Hematol. 2001, 38, 57-62.
12. Malcovati, L. Impact of Transfusion Dependency and Secondary Iron Overload on the Survival of Patients with Myelodysplastic Syndromes. Leukemia Res. 2007, 31, S2-S6.
13. Angelucci, E.; Brittenham, G. M.; McLaren, C. E.; Ripalti, M.; Baronciani, D.; Giardini, C.; Galimberti, M.; Polchi, P.; Lucarelli, G. Hepatic Iron Concentration and Total Body Iron Stores in Thalassemia Major. N. Engl. J. Med. 2000, 343, 327-331.
14. Bonkovsky, H. L.; Lambrecht, R. W. Iron-Induced Liver Injury. Clin. Liver Dis. 2000, 4, 409-429, yl-vii.
15. Peitrangelo, A. Mechanism of Iron Toxicity. Adv. Exp. Med. Biol., 2002, 509, 19-43.
16. Cario, H.; Holl, R. W.; Debatin, K. M.; Kohne, E. Insulin Sensitivity and β-Cell Secretion in Thalassemia Major with Secondary Haemochromatosis: Assessment by Oral Glucose Tolerance Test. Eur. J. Pediatr. 2004, 162, 139-146.
17. Wojcik, J. P.; Speechley, M. R.; Kertesz, A. E.; Chakrabarti, S.; Adams, P. C. Natural History of C282Y Homozygotes for Haemochromatosis. Can. J. Gastroenterol. 2002, 16, 297-302.
18. Brittenham, G. M. Disorders of Iron Metabolism: Iron Deficiency and Overload. In Hematology: Basic Principles and Practice, 3d ed.; Hoffman, R., Benz, E. J., Shattil, S. J. Furie, B., Cohen, H. J., et al., Eds.; Churchill Livingstone: New York, 2000; pp. 397-428.
19. Brittenham, G. M.; Griffith, P. M.; Nienhuis, A. W.; McLaren, C. E.; Young, N. S.; Tucker, E. E.; Allen, C. J.; Farrell, D. E.; Harris J. W. Efficacy of Deferoxamine in Preventing Complications of Iron Overload in Patients with Thalassemia Major. N. Engl. J. Med. 1994, 331, 567-573.
20. Zurlo, M. G.; De Stefano, P.; Borgna-Pignatti, C.; Di Palma, A.; Piga, A.; Melevendi, C.; Di Gregorio, F.; Burattini, M. G.; Terzoli, S. Survival and Causes of Death in Thalassemia Major. Lancet 1989, 2, 27-30.
21. http://www.pharma.us.novartis.com/product/pi/pdf/desferal.pdf
22. Hoffbrand, A. V.; Al-Refaie, F.; Davis, B.; Siritanakatkul, N.; Jackson, B. F. A.; Cochrane, J.; Prescott, E.; Wonke, B. Long-Term Trial of Deferiprone in 51 Transfusion-Dependent Iron Overloaded Patients. Blood 1998, 91, 295-300.
23. Olivieri, N. F. Long-Term Therapy with Deferiprone. Acta Haematol. 1996, 95, 37-48.
24. Olivieri, N. F.; Brittenham, G. M.; McLaren, C. E.; Templeton, D. M.; Cameron, R. G.; McClelland, R. A.; Burt, A. D.; Fleming, K. A. Long-Term Safety and Effectiveness of Iron-Chelation Therapy with Deferiprone for Thalassemia Major. N. Engl. J. Med. 1998, 339, 417-423.
25. Richardson, D. R. The Controversial Role of Deferiprone in the Treatment of Thalassemia. J. Lab. Clin. Med. 2001, 137, 324-329.
26. Nisbet-Brown, E.; Olivieri, N. F.; Giardina, P. J.; Grady, R. W.; Neufeld, E. J.; Sechaud, R.; Krebs-Brown, A. J.; Anderson, J. R.; Alberti, D.; Sizer, K. C.; Nathan, D. G. Effectiveness and Safety of ICL670 in Iron-Loaded Patients with Thalassaemia: A Randomised, Double-Blind, Placebo-Controlled, Dose-Escalation Trial. Lancet 2003, 361, 1597-1602.
27. Galanello, R.; Piga, A.; Alberti, D.; Rouan, M.-C.; Bigler, H.; Séchaud, R. Safety, Tolerability, and Pharmacokinetics of ICL670, a New Orally Active Iron-Chelating Agent in Patients with Transfusion-Dependent Iron Overload Due to β-Thalassemia. J. Clin. Pharmacol. 2003, 43, 565-572.
28. Cappellini, M. D. Iron-Chelating Therapy with the New Oral Agent ICL670 (Exjade). Best Pract. Res. Clin. Haematol. 2005, 18, 289-298.
29. Exjade Prescribing Information. http://www.pharma.us.novartis.com/product/pi/pdf/exjade.pdf (December 2007).
30. Galanello, R.; Formi, G.; Jones, A.; Kelly, A.; Willemsen, A.; He, X.; Johnston, A.; Fuller, D.; Donovan, J.; Piga, A. A Dose Escalation Study of the Pharmacokinetics, Safety & Efficacy of Deferitrin, an Oral Iron Chelator in Beta Thalassaemia Patients. ASH Annu. Meet. Abstr. 2007, 110, 2669.
31. Pippard, M. J. Desferrioxamine-Induced Iron Excretion in Humans. Bailliere's Clin. Haematol. 1989, 2, 323-343.
32. Giardina, P. J.; Grady, R. W. Chelation Therapy in β-Thalassemia: An Optimistic Update. Semin. Hematol. 2001, 38, 360-366.
33. Olivieri, N. F.; Brittenham, G. M. Iron-Chelating Therapy and the Treatment of Thalassemia. Blood 1997, 89, 739-761.
34. Bergeron, R. J.; Wiegand, J.; McManis, J. S.; Vinson, J. R. T.; Yao, H.; Bharti, N.; Rocca, J. R. (S)-4,5-Dihydro-2-(2-hydroxy-4-hydroxyphenyl)-4-methyl-4-thiazolecarboxylic Acid Polyethers: A Solution to Nephrotoxicity. J. Med. Chem. 2006, 49, 2772-2783.
35. Brittenham, G. M. Pyridoxal Isonicotinoyl Hydrazone. Effective Iron Chelation after Oral Administration. Ann. NY. Acad. Sci. 1990, 612, 315-326.
36. Naegeli, H.-U.; Zähner, H. Metabolites of Microorganisms. Part 193. Ferrithiocin. Helv. Chim. Acta 1980, 63, 1400-1406.
37. Hahn, F. E.; McMurry, T. J.; Hugi, A.; Raymond, K. N. Coordination Chemistry of Microbial Iron Transport. 42. Structural and Spectroscopic Characterization of Diastereomeric Cr(III) and Co(III) Complexes of Desferriferrithiocin. J. Am. Chem. Soc. 1990, 112, 1854-1860.
38. Anderegg, G.; Räber, M. Metal Complex Formation of a New Siderophore Desferrithiocin and of Three Related Ligands. J. Chem. Soc., Chem. Commun. 1990, 1194-1196.
39. Bergeron, R. J.; Wiegand, J.; Dionis, J. B.; Egli-Karmakka, M.; Frei, J.; Huxley-Tencer, A.; Peter, H. H. Evaluation of Desferrithiocin and Its Synthetic Analogues as Orally Effective Iron Chelators. J. Med. Chem. 1991, 34, 2072-2078.
40. Bergeron, R. J.; Streiff, R. R.; Wiegand, J.; Vinson, J. R. T.; Luchetta, G.; Evans, K. M.; Peter, H.; Jenny, H.-B. A Comparative Evaluation of Iron Clearance Models. Ann. N.Y. Acad. Sci. 1990, 612, 378-393.
41. Bergeron, R. J.; Streiff, R. R.; Creary, E. A.; Daniels, R. D., Jr.; King, W.; Luchetta, G.; Wiegand, J.; Moerker, T.; Peter, H. H. A Comparative Study of the Iron-Clearing Properties of Desferrithiocin Analogues with Desferrioxamine B in a Cebus Monkey Model. Blood 1993, 81, 2166-2173.
42. Bergeron, R. J.; Wiegand, J.; McManis, J. S.; McCosar, B. H.; Weimar, W. R.; Brittenham, G. M.; Smith, R. E. Effects of C-4 Stereochemistry and C-4′ Hydroxylation on the Iron Clearing Efficiency and Toxicity of Desferrithiocin Analogues. J. Med. Chem. 1999, 42, 2432-2440.
43. Bergeron, R. J.; Wiegand, J.; McManis, J. S.; Bussenius, J.; Smith, R. E.; Weimar, W. R. Methoxylation of Desazadesferrithiocin Analogues: Enhanced Iron Clearing Efficiency. J Med. Chem. 2003, 46, 1470-1477.
44. Bergeron, R. J.; Wiegand, J.; Weimar, W. R.; Vinson, J. R. T.; Bussenius, J.; Yao, G. W.; McManis, J. S. Desazadesmethyldesferrithiocin Analogues as Orally Effective Iron Chelators. J. Med. Chem. 1999, 42, 95-108.
45. Bergeron, R. J.; Wiegand, J.; McManis, J. S.; Bharti, N. The Design, Synthesis, and Evaluation of Organ-Specific Iron Chelators. J. Med. Chem. 2006, 49, 7032-7043.
46. Bergeron, R. J.; Wiegand, J.; Bharti, N.; Singh, S.; Rocca, J. R. Impact of the 3,6,9-Trioxadecyloxy Group on Desazadesferrithiocin Analogue Iron Clearance and Organ Distribution. J. Med. Chem. 2007, 50, 3302-3313.
47. Bergeron, R. J.; Wiegand, J.; McManis, J. S.; Bharti, N.; Singh, S. Design, Synthesis, and Testing of Non-Nephrotoxic Desazadesferrithiocin Polyether Analogues. J. Med. Chem. 2008, 51, 3913-3923.
48. Bergeron, R. J.; Bharti, N.; Wiegand, J.; McManis, J. S.; Yao, H.; Prokai, L. Polyamine Vectored Iron Chelators: The Role of Charge. J. Med. Chem. 2005, 48, 4120-4137.
49. Brunner, H; Gruber, N. Carboplatin-containing Porphyrin-platinum Complexes as Cytotoxic and Phototoxic Antitumor Agents. Inorg. Chim. Acta, 2004, 357, 4423-4451.
50. Kitto, H. J.; Schwartz, E.; Nijemeisland M.; Koepf M.; Cornelissen, J. J. L. M.; Rowan, A. E.; Nolte, R. J. M. Post-modification of Helical Dipeptido Polyisocyanides Using the ‘Click’ Reaction. J. Mater. Chem., 2008, 18, 5615-5624.
51. Bergeron, R. J.; Wiegand, J.; Weimar, W. R.; McManis, J. S.; Smith, R. E.; Abboud, K. A. Iron Chelation Promoted by Desazadesferrithiocin Analogues: An Enantioselective Barrier. Chirality 2003, 15, 593-599.
52. Sangster, J. Octanol-Water Partition Coefficients: Fundamentals and Physical Chemistry; John Wiley and Sons: West Sussex, England, 1997; Vol. 2.
53. White, G. P.; Jacobs, A.; Grady, R. W.; Cerami, A. The Effect of Chelating Agents on Iron Mobilization in Chang Cell Cultures. Blood 1976, 48, 923-929.
54. White, G. P.; Bailey-Wood, R.; Jacobs, A. The Effect of Chelating Agents on Cellular Iron Metabolism. Clin. Sci. Mol. Med. 1976, 50, 145-52.
55. Bergeron, R. J.; McManis, J. S.; Weimar, W. R.; Wiegand, J. Eiler-McManis, E. Iron Chelators and Therapeutic Uses. In: Abraham DA, editor. Burger's Medicinal Chemistry. 6th. Wiley; New York: 2003. pp. 479-561.
56. http://www.torpac.com/Torpac%20Rat%20Gpig%20Pricing.pdf
57. SHELXTL6; Bruker-AXS: Madison, Wis., 2000.
58. Bergeron, R. J.; Wiegand, J.; Ratliff-Thompson, K.; Weimar, W. R. The Origin of the Differences in (R)- and (S)-Desmethyldesferrithiocin: Iron-Clearing Properties. Ann. N Y. Acad. Sci. 1998, 850, 202-216.
59. Bergeron, R. J.; Streiff, R. R.; Wiegand, J.; Luchetta, G.; Creary, E. A.; Peter, H. H. A Comparison of the Iron-Clearing Properties of 1,2-Dimethyl-3-Hydroxypyrid-4-one, 1,2-Diethyl-3-Hydroxypyrid-4-one, and Deferoxamine. Blood 1992, 79, 1882-1890.
60. Wood, J. K.; Milner, P. F.; Pathak, U. N. The Metabolism of Iron-Dextran Given As a Total-Dose Infusion to Iron Deficient Jamaican Subjects. Br. J. Hamaetol. 1968, 14, 119-129.
61. Bergeron, R. J.; Wiegand, J.; Brittenham, G. M. HBED: A Potential Alternative to Deferoxamine for Iron-Chelating Therapy. Blood 1998, 91, 1446-1452.
62. Bergeron, R. J.; Wiegand, J.; Wollenweber, M.; McManis, J. S.; Algee, S. E.; Ratliff-Thompson, K. Synthesis and Biological Evaluation of Naphthyldesferrithiocin Iron Chelators. J. Med. Chem. 1996, 39, 1575-1581.

1. A compound of the formula (I) 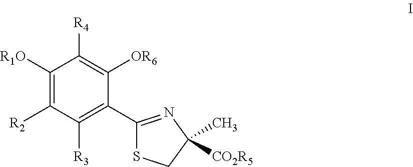
wherein
R₁ is —[(CH₂)ₙ—O]ₓ—R′;
R₂, R₃, and R₄ are each independently —H, an alkyl group, or —OR₇;
R₅ is —H or an alkyl group;
R₆ is —H, an alkyl group, an O-protecting group, or an acyl group
each R₇ is independently —H, an alkyl group, an O-protecting group, or an acyl group;
R′ is —H, an alkyl group, an O-protecting group, or an acyl group;
each n is 2;
x is 1 or 2;
or a salt, solvate, or hydrate thereof;
with the proviso that the compound is not of formula (II)

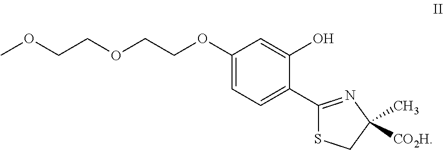

2. The compound of claim 1, wherein R₂, R₃, and R₄ are each independently —H, a C₁₋₆ alkyl group, or —OR₇; R₆ is —H, an O-protecting group, or an acyl group.

3. (canceled)

4. The compound of claim 1, wherein x is 2, and R₂, R₃, R₄ and R₆ are hydrogen.

5-16. (canceled)

17. The compound of claim 1, wherein R₆ is hydrogen.

18-21. (canceled)

22. The compound of claim 1, wherein R₂, R₃, R₄ and R₆ are hydrogen.

23. The compound of claim 1, wherein x is 1, and R₂, R₃, R₄ and R₆ are hydrogen.

24. The compound of claim 1, R′ is a C₁₋₄ alkyl group.

25. (canceled)

26. The compound of claim 1, wherein R′ is a methyl.

27. (canceled)

28. The compound of claim 1, wherein x is 2.

29. (canceled)

30. The compound of claim 1, wherein x is 2, and R′ is methyl.

31. The compound of claim 1, wherein the compound is:

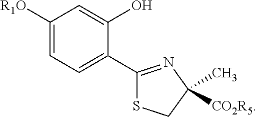

32. The compound of claim 1, wherein the compound is:

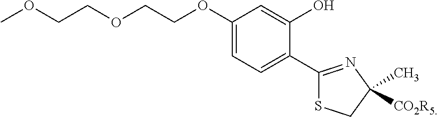

33. (canceled)

34. The compound of claim 1, wherein the compound is:

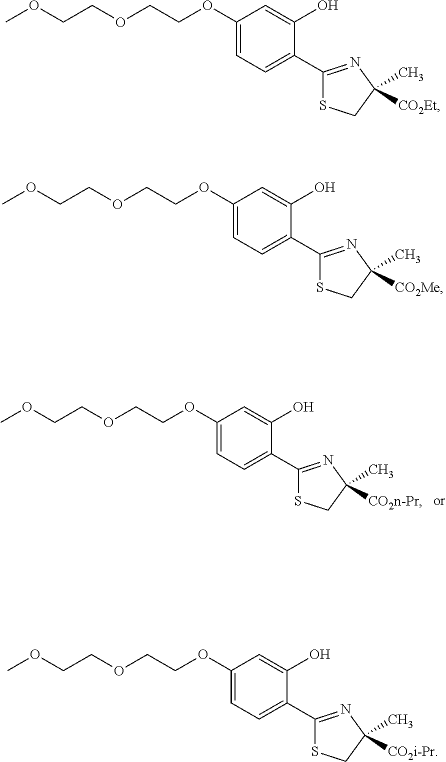

35-37. (canceled)

38. The compound of claim 1, wherein the compound is a solid.

39. The compound of claim 1, wherein the compound is crystalline solid.

40-50. (canceled)

51. A solid or crystalline form of a compound of formula: 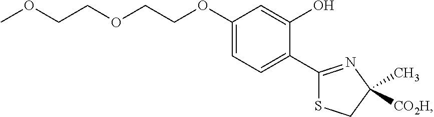
or a salt, solvate, or hydrate thereof.

52-57. (canceled)

58. A pharmaceutical composition comprising a compound of claim 1 and a pharmaceutically acceptable excipient.

59. (canceled)

60. A method of treating a pathological condition responsive to chelation of a trivalent metal in a subject, comprising administering to the subject a therapeutically or prophylactically effective amount of claim 1.

61. (canceled)

62. The method of claim 60, wherein the trivalent metal is iron.

63-77. (canceled)

78. A method of reducing oxidative stress in a subject in need of treatment comprising administering to the subject a therapeutically effective amount of a compound of claim 1.

79.-82. (canceled)
